(19) United States
(12) Patent Application Publication
(10) Pub. No.: US 2017/0266292 A1
(43) Pub. Date: Sep. 21, 2017

(54) LIPIDIC COMPOUND-TELODENDRIMER HYBRID NANOPARTICLES AND METHODS OF MAKING AND USES THEREOF

(71) Applicant: The Research Foundation for The State University of New York, Syracuse, NY (US)

(72) Inventors: Juntao Luo, Jamesville, NY (US); Xu Wang, Syracuse, NY (US); Changying Shi, Jamesville, NY (US); Alexa Bodman, Syracuse, NY (US); Walter Hall, Fayetteville, NY (US)

(21) Appl. No.: 15/464,918

(22) Filed: Mar. 21, 2017

Related U.S. Application Data
(60) Provisional application No. 62/311,005, filed on Mar. 21, 2016.

(51) Int. Cl. A61K 47/34

(52) U.S. Cl. CPC A61K 47/34

(57) ABSTRACT

Lipidic compound (lipidic molecule)-telodendrimer hybrid nanoparticles. For example, the lipidic compound-telodendrimer hybrid nanoparticles are lipid/lipidoid-telodendrimer hybrid nanoparticles. The nanoparticles can comprise a plurality of lipidic molecules (e.g., lipid molecules, lipidoid molecules, or mixtures of different lipid molecules or different lipidoid molecules). The hybrid nanoparticles can comprise one or more lipid or lipidoid and one or more telodendrimer. The hybrid nanoparticles can also comprise cholesterol. In various examples, the hybrid nanoparticles also comprise a small molecule, peptide, protein, or a combination thereof. In various examples, lipid-telodendrimer hybrid nanoparticles comprising one or more small molecules or lipidoid-telodendrimer hybrid nanoparticles comprising one or more protein(s) and/or peptide(s) are used in methods of small-molecule or protein/peptide delivery.

### CROSS REFERENCE TO RELATED APPLICATIONS

This application claims priority to U.S. Provisional Application No. 62/311,005, filed on Mar. 21, 2016, the disclosure of which is hereby incorporated by reference.

### STATEMENT REGARDING FEDERALLY SPONSORED RESEARCH

This invention was made with Government support under grant nos. CA140449 and EB019607 awarded by the National Institutes of Health. The Government has certain rights in the invention.

### BACKGROUND OF THE DISCLOSURE

Protein therapy has become a promising approach for the treatment of human disease since the introduction of the first human recombinant protein therapeutic (Humulin) over 30 years ago. Currently, more than 130 proteins are approved for clinical use by the US Food and Drug Administration. Many more therapeutic proteins are in development that would play an important role in almost every field of medicine. The majority of the protein therapeutics target to the extracellular receptors. With the advance of the modern techniques for protein recombination and antibody engineering, it is feasible to develop specific antibodies against many intracellular targets, as applied in biochemical detection and pathological diagnosis. The application of protein therapeutics directly against the intracellular targets is still in its infancy due to their poor permeability across cell membrane. The properties preventing their cellular uptake include surface charge distributions, large sizes and vulnerable tertiary structures. Therefore, the development of feasible ways for effective intracellular delivery of such proteins to their intracellular targets is expected to open a new horizon for protein therapeutics in disease treatments.

Nanocarriers originally developed for delivery of small-molecule drugs with enhanced targeting effects and reduced side effects have been identified as candidates for intracellular protein delivery. These nanocarriers mainly include liposomes, nanogels, polymeric nanoparticles (NPs), and inorganic nanomaterials. Some of them have been investigated for intracellular protein delivery for the treatment of cancers such as breast cancer, lung cancer, etc. Glioblastoma multiforme (GBM) is the most common and most aggressive malignant primary brain tumor in humans, and it is extremely difficult to treat because (i) it can achieve infiltrative growth by differentiating into the intricate network of blood vessels resulting in a frequently occurring reappearance after resection, and (ii) the blood-brain barrier (BBB) formed by the brain capillary endothelium in the central nervous system excludes from the brain almost all of the large-molecule neurotherapeutics and most small-molecule drugs. Current treatment with surgical resection, temozolomide, and radiation has only modestly increased overall survival with a one-year survival of 45%.

### SUMMARY OF THE DISCLOSURE

In an aspect, the present disclosure provides lipidic compound (lipidic molecule)-telodendrimer hybrid nanoparticles. For example, the lipidic compound (lipidic molecule)-telodendrimer hybrid nanoparticles are lipid/lipidoid-telodendrimer hybrid nanoparticles. The nanoparticles can comprise a plurality of lipidic molecules (e.g., lipid molecules, lipidoid molecules, or mixtures of different lipid molecules or different lipidoid molecules). The hybrid nanoparticles can comprise one or more lipid or lipidoid and one or more telodendrimer. The hybrid nanoparticles can also comprise cholesterol. The hybrid nanoparticles have a hydrophobic domain (e.g., provided by the lipid or lipidoid) and a hydrophilic domain (provided by the telodendrimer). In various examples, the hybrid nanoparticles also comprise a small molecule, peptide, protein, or a combination thereof.

Lipid-telodendrimer hybrid nanoparticles can comprise a variety of lipids. Suitable lipids comprise one or more hydrophilic head groups with neutral charges (e.g., phosphocholine or other zwitterionic moieties) and one or more hydrophobic moieties (e.g., two aliphatic carbon chains). For example, individual aliphatic carbon chains have 4 to 100 carbons, including all integer number of carbons and ranges therebetween. Examples of suitable lipids are commercially available or can be made using methods known in the art.

Lipidoid-telodendrimer hybrid nanoparticles can comprise a variety of lipidoids. Lipidoids can comprise one or more positively charged groups (e.g., primary amine, secondary amine, tertiary amine, or guanidine groups) and one or more hydrophobic moieties (e.g., single aliphatic or double aliphatic carbon chains). For example, the lipidoid has single aliphatic or double aliphatic carbon chains comprising 2 to 50 carbons, including all integer number of carbons and ranges therebetween. A lipidoid can be an amphiphilic molecule comprising positively charged or negatively charged head groups conjugated with hydrophobic molecules. Examples of suitable lipidoids are commercially available or can be made using methods known in the art.

Lipidic compound (lipidic molecule)-telodendrimer hybrid nanoparticles can comprise a variety of telodendrimers. For example, are functional segregated telodendrimers having, for example, two or three functional segments. The telodendrimers can be tri-block telodendrimers with segregated functional regions. Telodendrimers can be referred to as, for example, G1, G2, or G3 telodendrimers. Examples of telodendrimers are shown in FIGS. 27-29.

In an aspect, the present disclosure provides methods of making lipidic compound (lipidic molecule)-telodendrimer hybrid nanoparticles (e.g., lipid/lipidoid-telodendrimer hybrid nanoparticles). The hybrid nanoparticles can be made using methods described herein. Also provided are methods of making protein/peptide or small-molecule loaded hybrid nanoparticles. The protein/peptide or small-molecule loaded hybrid nanoparticles can be made using methods described herein.

In an aspect, the present disclosure provides methods of using lipidic compound (lipidic molecule)-telodendrimer hybrid nanoparticles (e.g., lipid/lipidoid-telodendrimer hybrid nanoparticles). For example, lipid-telodendrimer hybrid nanoparticles comprising one or more small molecules or lipidoid-telodendrimer hybrid nanoparticles comprising one or more protein(s) and/or peptide(s) can be used in methods of small-molecule or protein/peptide delivery. The lipidic compound (lipidic molecule)-telodendrimer hybrid nanoparticles (e.g., lipid/lipidoid-telodendrimer hybrid nanoparticles) of the present disclosure can be used to treat any disease requiring the administration of a drug, such as by sequestering a hydrophobic drug in the interior of a hybrid nanoparticle or by stabilizing a protein with one or more hybrid nanoparticle.

### BRIEF DESCRIPTION OF THE FIGURES

For a fuller understanding of the nature and objects of the disclosure, reference should be made to the following detailed description taken in conjunction with the accompanying figures.

FIG. 1 shows particle size (a) and ζ-potential (b) of lipidoid-PEG^{5k}CA₄-L-CHO₄ NPs at different L/T mass ratios and BSA-incorporated NPs in PBS at a NP concentration of 0.2 mg/mL, and TEM images (c-e) and particle size analysis (f-h) of lipidoid-PEG^{5k}CA₄-L-CHO₄ NPs at L/T mass ratios of 30/70 (c,f), 50/50 (d,g) and 80/20 (e,h).

FIG. 2 shows (a,b) loading capacities of lipidoid-PEG^{5k}CA₄-L-CHO₄ NPs at different L/T mass ratios (30/70, 50/50 and 80/20) for FITC-BSA determined by agarose gel retention assay under SYBR Green (a) and Coomassie Blue (b) modes from free FITC-BSA (lane 1), FITC-BSA-loaded 30/70 NPs (lane 2), FITC-BSA-loaded 50/50 NPs (lane 3), FITC-BSA-loaded 80/20 NPs (lane 4), 30/70 NPs (lane 5), 50/50 NPs (lane 6), and 80/20 NPs (lane 7). (c) Release profiles of free FITC-BSA and FITC-BSA loaded in lipidoid-PEG^{5k}CA₄-L-CHO₄ NPs at different L/T mass ratios from agarose gels.

FIG. 3 shows (a,b) Confocal laser fluorescence microscopy images of U87 cells incubated at 37° C. for 2 h (h=hour(s)) with free FITC-BSA (a), and FITC-BSA-incorporated lipidoid-PEG^{5k}CA₄-L-CHO₄ NPs at an L/T ratio of 50/50 and a FITC-BSA/NP ratio of 1/10 by weight (b). The images were taken at a magnification of 60× with a final FITC concentration of approximately 3 μg/mL. (c) Mean fluorescence intensity in U87 and HT-29 cells following a 2 h incubation at 37° C. with free FITC-BSA, and FITC-BSA-incorporated lipidoid-PEG^{5k}CA₄-L-CHO₄ NPs at different L/T mass ratios.

FIG. 4 shows cell viability assays on GBM cell lines of U87 (a), LN229 (b) and U138 (c) after a 72 h continuous incubation at 37° C. for free DT₃₉₀, lipidoid-PEG^{5k}CA₄-L-CHO₄ NPs with different L/T mass ratios (30/70, 50/50 and 80/20), and DT₃₉₀-loaded hybrid NPs at a DT₃₉₀ loading ratio of 1/10 (DT₃₉₀/NP, w/w). The black arrow indicates the highest NP concentration used for DT₃₉₀ delivery.

FIG. 5 shows distributions in brain sections containing U87 tumors indicated by dotted circles (a) and in intracranial U87 tumors at the cellular level (b) of free Cy5-BSA, and Cy5-BSA incorporated in lipidoid-PEG^{5k}CA₄-L-CHO₄ NPs with different L/T mass ratios (50/50 and 80/20) at a loading ratio of 1/10 (protein/NP, w/w) determined by confocal microscopy. The cell nuclei in (b) were stained with DAPI.

FIG. 6 shows (a) typical bioluminescence images of mice injected with intracranial U87 tumors treated with different formulations taken using IVIS 50 at different days post injection. (b) DT₃₉₀-incorporated NP delivery suppresses tumor growth on mice injected with intracranial U87 tumors. *P<0.05 as compared to each control group. (c,d) Histological images of the H&E (c) and TUNEL immunofluorescence staining (d) assays for intracranial U87 tumor tissues after treatment with PBS, free DT₃₉₀, BSA-incorporated NPs and DT₃₉₀-incorporated NPs. The cell nuclei in (d) were stained with DAPI (blue).

FIG. 7 shows DLS particle size of Mcl-1 inhibitor-loaded lipidoid-telodendrimer hybrid nanoparticles (L/T ratio of 80/20, w/w) at a drug loading ratio of 1/10 (drug/nanoparticle, w/w) in PBS.

FIG. 8 shows TEM images of the hybrid NPs of DPPC and PEG^{5k}(Arg-L-CHO)₄ at blending mass ratios of 1:1 (a) and 5:1 (b).

FIG. 9 shows TEM images of the hybrid NPs of DMPC and PEG^{5k}CA₄-L-Rh₄ at a blending mass ratio of 5:1 before (a) and after (b) loading of Amb at a loading ratio of 1/10 (Amb/NP, w/w).

FIG. 10 shows schematic illustration of the fabrication of protein-loaded lipidoid-telodendrimer hybrid nanoparticles and the intracellular delivery of protein therapeutics within the nanoparticles to treat brain cancers.

FIG. 11 shows ¹H NMR spectrum of the lipidoid made from 1,2-epoxyhexadecane and N,N′-dimethyl-1,3-propanediamine in CDCl₃. Inset shows the chemical structure of the lipidoid.

FIG. 12 shows MALDI-TOF mass spectrum of the lipidoid.

FIG. 13 shows Hydrodynamic size distribution of the lipidoid in PBS (1×) at a particle concentration of 0.2 mg/mL.

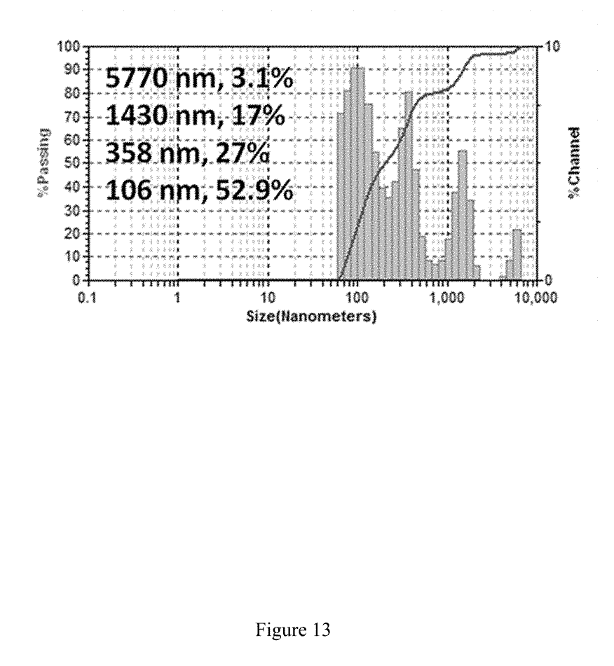

FIG. 14 shows hydrodynamic size distributions of lipidoid-PEG^{5k} CA₈ hybrid nanoparticles (lipidoid/telodendrimer=50/50, w/w) and BSA loaded nanoparticles (BSA/nanoparticle=1/10, w/w) in PBS (1×) at a nanoparticle concentration of 0.2 mg/mL.

FIG. 15 shows hydrodynamic size distributions of lipidoid-PEG^{5k}CHO₈ hybrid nanoparticles (lipidoid/telodendrimer=50/50, w/w) and BSA loaded nanoparticles (BSA/nanoparticle=1/10, w/w) in PBS (1×) at a nanoparticle concentration of 0.2 mg/mL.

FIG. 16 shows hydrodynamic size distributions of lipidoid-PEG^{5k}CA₄CHO₈ hybrid nanoparticles (lipidoid/telodendrimer=50/50, w/w) and BSA loaded nanoparticles (BSA/nanoparticle=1/10, w/w) in PBS (1×) at a nanoparticle concentration of 0.2 mg/mL.

FIG. 17 shows hydrodynamic size distributions of lipidoid-PEG^{5k}CA₄-L-CHO₈ hybrid nanoparticles (lipidoid/telodendrimer=50/50, w/w) and BSA loaded nanoparticles (BSA/nanoparticle=1/10, w/w) in PBS (1×) at a nanoparticle concentration of 0.2 mg/mL.

FIG. 18 shows hemolytic property of lipidoid-telodendrimer hybrid nanoparticles with a mass ratio of lipidoid to telodendrimer of 50/50 at different time points after the diluted RBC suspension was mixed with the nanoparticles.

FIG. 19 shows cell viability assays on U87 cells after a 72 h continuous incubation at 37° C. for lipidoid-telodendrimer hybrid nanoparticles at a constant ratio of 50:50 (lipidoid/telodendrimer, w/w), in comparison with PEI. No significant toxicity for the lipidoid-telodendrimer hybrid nanoparticles was observed at 5 μg/mL concentration. The lipidoid-telodendrimer hybrid nanoparticles have lower cytotoxicities than that for a common transfection agent of PEI.

FIG. 20 shows hydrodynamic size distributions of lipidoid-PEG^{5k}CA₄-L-CHO₄ hybrid nanoparticles with different mass ratios of lipidoid to PEG^{5k}CA₄-L-CHO₄ in PBS (1×) at a particle concentration of 0.2 mg/mL.

FIG. 21 shows hydrodynamic size distributions of BSA-incorporated lipidoid-PEG^{5k}CA₄-L-CHO₄ hybrid nanoparticles (BSA/nanoparticle=1/10, w/w) with different mass ratios of lipidoid to PEG^{5k}CA₄-L-CHO₄ in PBS (1×) at a particle concentration of 0.2 mg/mL.

FIG. 22 shows hemolytic property of lipidoid-PEG^{5k}CA₄-L-CHO₄ hybrid nanoparticles with different mass ratios of lipidoid to telodendrimer at different time points after the diluted RBC suspension was mixed with the nanoparticles.

FIG. 23 shows cell viability assays on U87 cells after a 72 h continuous incubation at 37° C. for lipidoid-PEG^{5k}CA₄-L-CHO₄ hybrid nanoparticles at different mass ratios of lipidoid to telodendrimer. No significant toxicity for the hybrid nanoparticles was observed at 5 μg/mL concentration while toxicity was observed at this concentration for lipidoid.

FIG. 24 shows TEM images (a-c) and particle size analysis (d-f) of BSA-loaded lipidoid-PEG^{5k}CA₄-L-CHO₄ hybrid nanoparticles at mass ratios of lipidoid to telodendrimer of 30/70 (a,d), 50/50 (b,e), and 80/20 (c,f). The loading ratio is 1/10 (BSA/nanoparticle, w/w).

FIG. 25 shows microscopy images of U87 cells incubated at 37° C. for 2 h with free FITC-BSA and FITC-BSA-incorporated lipidoid-PEG^{5k}CA₄-L-CHO₄ hybrid nanoparticles at different mass ratios of lipidoid to telodendrimer. The mass ratio of FITC-BSA to nanoparticle is 1:10, and the final FITC concentration is approximately 1 μg/mL. The images were taken at a magnification of 32×.

FIG. 26 shows microscopy images of HT-29 cells incubated at 37° C. for 2 h with free FITC-BSA and FITC-BSA-incorporated lipidoid-PEG^{5k}CA₄-L-CHO₄ hybrid nanoparticles at different mass ratios of lipidoid to telodendrimer. The mass ratio of FITC-BSA to nanoparticle is 1:10, and the final FITC concentration is approximately 1 μg/mL. The images were taken at a magnification of 32×.

FIG. 27 shows chemical structures of a lipidoid made from 1,2-epoxyhexadecane and N,N′-dimethyl-1,3-propanediamine, and four telodendrimers with varying architectures and compositions containing CA and/or CHO groups.

FIG. 28 shows chemical structure of PEG^{5k}(Arg-L-CHO)₄.

FIG. 29 shows chemical structure of PEG^{5k}CA₄-L-Rh₄.

### DETAILED DESCRIPTION OF THE DISCLOSURE

The present disclosure provides lipidic compound (lipidic molecule)-telodendrimer hybrid nanoparticles, which can comprise a small molecule, peptide, protein, or a combination thereof, and uses of the nanoparticles. Examples of lipidic compounds (also referred to herein as lipidic molecules) include, but are not limited to, lipids and lipidoids. For example, the nanoparticles are used for drug delivery. Examples of drugs include, but are not limited to, small molecule drugs, peptides, and proteins.

In this disclosure, we have developed a novel strategy to form stable hybrid nanoparticle using rationally designed telodendrimers and lipidic compounds for intracellular protein therapeutics delivery and for membrane active drug delivery. Our hybrid nanoformulation can efficiently deliver toxin to treat GBM efficiently.

Ranges of values are disclosed herein. The ranges set out a lower limit value and an upper limit value. Unless otherwise stated, the ranges include all values to the magnitude of the smallest value (either lower limit value or upper limit value) and ranges between the values of the stated range.

Definitions. As used herein, the term “telodendrimer” refers to a linear-dendritic copolymer, containing an optional hydrophilic segment (i.e., PEG moiety) and one or more chemical moieties covalently bonded to one or more end groups of the dendron. Suitable moieties include, but are not limited to, hydrophobic groups, hydrophilic groups, amphiphilic compounds, and drugs. Different moieties may be selectively installed at selected end groups using orthogonal protecting group strategies.

As used herein, the term “moiety” refers to a part (substructure) or functional group of a molecule that is part of the telodendrimer structure. For example,

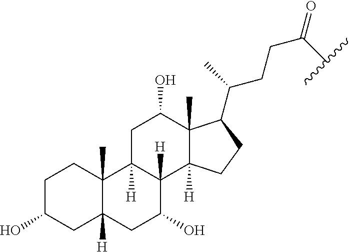

refers to a cholic acid moiety,

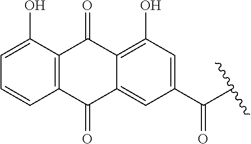 [SMILES: *C(=O)c1cc(O)c2c(c1)C(=O)c1cccc(O)c1C2=O]

refers to a rhein moiety,

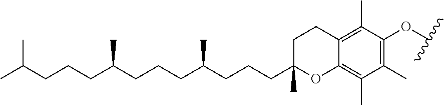 [SMILES: *Oc1c(C)c(C)c2c(c1C)CC[C@@](C)(CCC[C@H](C)CCC[C@H](C)CCCC(C)C)O2]

refers to a vitamin E moiety. Connections between a moiety and adjacent moieties (e.g., to one or more PEG moiety, X moiety, D¹ moiety, D² moiety, L¹ moiety, L² moiety, R¹ moiety, and/or R² moiety) can be direct covalent bonds (e.g., where an amide bond links a moiety to an adjacent moiety), or short linking groups such as, for example, carbonyls (e.g., where a carbamate links a moiety to an adjacent moiety). Accordingly, a moiety may comprise an additional functional group or a short linking group). For example, cholesterol moieties can refer to

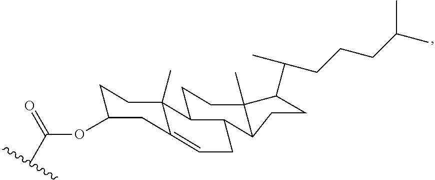 [SMILES: *C(=O)OC1CCC2(C)C(=CCC3C2CCC2(C)C(C(C)CCCC(C)C)CCC32)C1]

where the cholesterol moiety comprises a carbonyl (in addition to the cholesterol base structure) added to accommodate a linkage between the nitrogen from the adjacent moiety and the alcohol from the cholesterol molecule.

As used herein, the terms “dendritic polymer” refer to branched polymers comprising a focal point, one or more branched monomer units, and one or more end groups. Monomers can be linked together to form arms (or “dendritic polymer”) extending from the focal point and terminating at end groups. The focal point of the dendritic polymer can be attached to other segments of the compounds of the disclosure, and the end groups may be further functionalized with additional chemical moieties.

As used herein, the terms “monomer” and “monomer unit” refer to monomers such as diamino carboxylic acids, dihydroxy carboxylic acids, hydroxyl amino carboxylic acids and monomer units derived from such monomers. A monomer or monomer unit is also referred to herein as a “branched monomer” or “branched monomer units.” The side chain of the monomer (e.g., the amino group in the side chain of a lysine moiety) can be covalently bonded to one or more monomers or monomer units. For example, the amino group on the side chain of lysine may be bonded to one or two additional monomer or branched monomer units (e.g., lysine moieties), which may further be bonded to additional monomer units. Examples of diamino carboxylic acid groups of the present disclosure include, but are not limited to, 2,3-diamino propanoic acid, 2,4-diaminobutanoic acid, 2,5-diaminopentanoic acid (ornithine), 2,6-diaminohexanoic acid (lysine), (2-aminoethyl)-cysteine, 3-amino-2-aminomethyl propanoic acid, 3-amino-2-aminomethyl-2-methyl propanoic acid, 4-amino-2-(2-aminoethyl) butyric acid and 5-amino-2-(3-aminopropyl) pentanoic acid. Examples of dihydroxy carboxylic acid groups of the present disclosure include, but are not limited to, glyceric acid, 2,4-dihydroxybutyric acid, glyceric acid, 2,4-dihydroxybutyric acid, 2,2-bis(hydroxymethyl)propionic acid, and 2,2-bis(hydroxymethyl)butyric acid. Examples of hydroxyl amino carboxylic acids include, but are not limited to, serine and homoserine. One of skill in the art will appreciate that other monomer units can be used in the present disclosure.

Monomers of the present disclosure can have a bond connectivity of, for example,

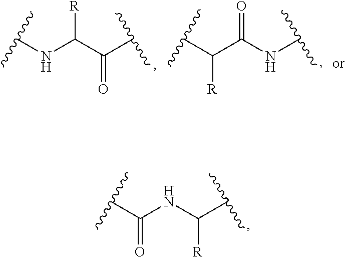

where R is a side chain of an amino acid moiety. For example, when a monomer is a lysine moiety, then the moiety structure can be, e.g., one of the following structures:

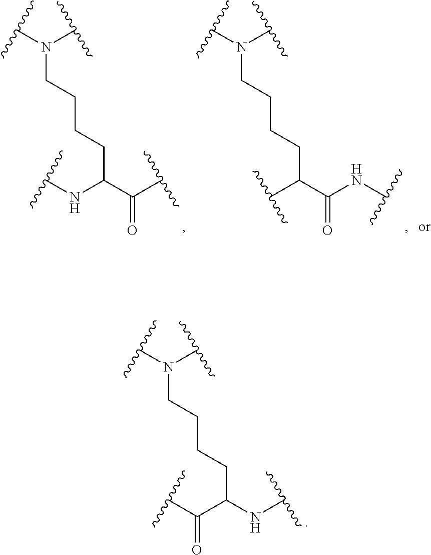 [SMILES: *NC(CCCCN(*)*)C(*)=O ; *NC(=O)C(*)CCCCN(*)* ; *NC(CCCCN(*)*)C(*)=O]

As used herein, the term “linker” refers to a chemical moiety that links (e.g., via covalent bonds) one segment of a dendritic conjugate to another segment of the dendritic conjugate. The types of bonds used to link the linker to the segments of the telodendrimers include, but are not limited to, amides, amines, esters, carbamates, ureas, thioethers, thiocarbamates, thiocarbonate, and thioureas. For example, the linker (L¹, L¹, L², and/or L³), individually at each occurrence in the telodendrimer, can be a polyethylene glycol moiety, polyserine moiety, polyglycine moiety, poly(serine-glycine) moiety, aliphatic amino acid moieties, 6-amino hexanoic acid moiety, 5-amino pentanoic acid moiety, 4-amino butanoic acid moiety, and beta-alanine moiety. The linker can also be a cleavable linker. In various examples, combinations of linkers can be used. For example, the linker can be an enzyme cleavable peptide moiety, disulfide bond moiety or an acid labile moiety. One of skill in the art will appreciate that other types of bonds can be used in the present disclosure. In various examples, the linker L, L¹, L², and/or L³ can be

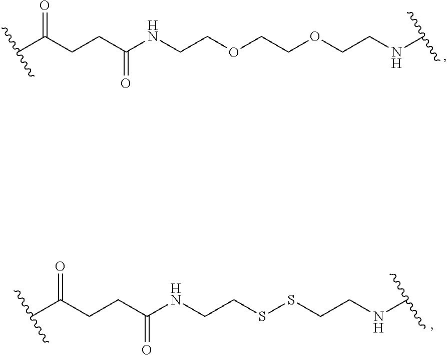 [SMILES: *NCCOCCOCCNC(=O)CCC(*)=O ; *NCCSSCCNC(=O)CCC(*)=O]

or a combination thereof.

As used herein, PEG group refers to polyethylene glycol. For example, the structure of PEG is

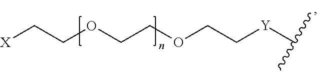

where X is selected from the group consisting of —NH₂, —OH, —SH, —COOH, —OMe, —N₃, —C═CH₂, or —≡CH, Y is selected from the group consisting of —C(═O)O—, —OC(═O)—, —OC(═O)NH—, —NHC(═O)—, —NHC(═O)O—, —NH—, —O—, —S—,

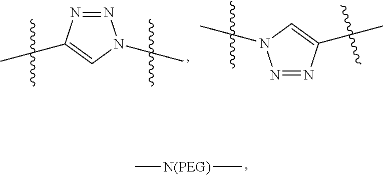

—NHCOLys(PEG)-, —NHCO[branched Lys(PEG)]ₙNH—, -Lys-, -Lys(PEG)-, -Lys(PEG)-Lys, -Lys(PEG)-Lys(PEG)-, Lys(PEG-Lys-Lys(PEG), and -Lys(PEG)-Lys(Lys(PEG)₂)-Lys- and n is the number of repeating unit in a range of 1 to 72736, including all integer values and ranges therebetween.

As used herein, the term “oligomer” refers to sixteen or fewer monomers, as described above, covalently linked together. The monomers may be linked together in a linear or branched fashion. The oligomer may function as a focal point for a branched segment of a telodendrimer.

As used herein, the term “hydrophobic group” refers to a chemical moiety that is water-insoluble or repelled by water. Examples of hydrophobic groups include, but are not limited to, long-chain alkanes and fatty acids, fluorocarbons, silicones, certain steroids such as, for example, cholesterol, and certain polymers such as, for example, polystyrene, polyisoprene, polylactide, polyglycolide, and polycaprolactone.

As used herein, the term “hydrophilic group” refers to a chemical moiety that is water-soluble or attracted to water. Examples of hydrophilic groups include, but are not limited to, alcohols, short-chain carboxylic acids, quaternary amines, sulfonates, phosphates, sugars, and certain polymers such as, for example, PEG.

As used herein, the term “amphiphilic compound” refers to a compound having both hydrophobic portions and hydrophilic portions. For example, the amphiphilic compounds of the present disclosure can have one hydrophilic part of the compound and one hydrophobic part of the compound, for example bile acids, cholic acids, riboflavin, chlorogenic acid, etc.

As used herein, the terms “treat”, “treating” and “treatment” refer to any indicia of success in the treatment or amelioration of an injury, pathology, condition, or symptom (e.g., pain), including any objective or subjective parameter such as abatement; remission; diminishing of symptoms or making the symptom, injury, pathology or condition more tolerable to the patient; decreasing the frequency or duration of the symptom or condition; or, in some situations, preventing the onset of the symptom or condition. The treatment or amelioration of symptoms can be based on any objective or subjective parameter; including, e.g., the result of a physical examination.

As used herein, the term “individual” refers to animals such as mammals. Suitable examples of mammals include, but are not limited to, primates (e.g., humans), cows, sheep, goats, horses, dogs, cats, rabbits, rats, mice, and the like. In an example, the individual is a human.

As used herein, the terms “therapeutically effective amount or dose” or “therapeutically sufficient amount or dose” or “effective or sufficient amount or dose” refer to a dose that produces therapeutic effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins). In sensitized cells, the therapeutically effective dose can often be lower than the conventional therapeutically effective dose for non-sensitized cells.

In an aspect, the present disclosure provides lipidic compound (lipidic molecule)-telodendrimer hybrid nanoparticles. For example, the lipidic compound (lipidic molecule)-telodendrimer hybrid nanoparticles are lipid/lipidoid-telodendrimer hybrid nanoparticles. The nanoparticles can comprise a plurality of lipidic molecules (e.g., lipid molecules, lipidoid molecules, or mixtures of different lipid molecules or different lipidoid molecules). The hybrid nanoparticles can comprise one or more lipid or lipidoid and one or more telodendrimer. The hybrid nanoparticles can also comprise cholesterol. The hybrid nanoparticles have a hydrophobic domain (e.g., provided by the lipid or lipidoid) and a hydrophilic domain (provided by the telodendrimer). In various examples, the hybrid nanoparticles also comprise a small molecule, peptide, protein, or a combination thereof.

Lipidic compounds (lipidic molecules) have one or more hydrophobic moieties (e.g., neutral and/or zwitterionic and/or charged (positively charged or negatively charged) moieties). Lipidic compounds (lipidic molecules) include, but are not limited to, lipids and lipidoids with one or more hydrophilic moiety (e.g., hydrophilic neutral moiety and/or hydrophilic charged moiety) and hydrophobic moiety.

Lipid-telodendrimer hybrid nanoparticles can comprise a variety of lipids. Suitable lipids comprise one or more hydrophilic head groups with neutral charges (e.g., phosphocholine or other zwitterionic moieties) and one or more hydrophobic moieties (e.g., two aliphatic carbon chains). For example, individual aliphatic carbon chains have 4 to 100 carbons, including all integer number of carbons and ranges therebetween. Examples of suitable lipids are commercially available or can be made using methods known in the art.

Suitable lipids can form stable lipid bilayer membranes. Without intending to be bound by any particular theory, it is considered that hydrophilic group(s) of a lipid stabilizes the bilayer nanodisc structure and a telodendrimer can stabilize a fragmented small piece of such bilayer membrane to form a nanodisc (e.g., a three-layered telodendrimer structure stabilizes the edges of a nanodisc efficiently and the PEG chain on telodendrimer prevents nanodisc stacking in a Z-direction). Cholesterol can be used to stabilize the membrane structure of a nanodisc. The two hydrophobic carbon chains in the lipids are important for the stability of bilayer membrane as well as the affinity with the telodendrimer in forming stable nanodisc structures. The two hydrophobic carbon chains in the lipids are generally required to have similar or the same chemical structure to form well-defined, stable nanodiscs together with the telodendrimers. Moreover, the hydrophobic groups and the disk-like morphology of the lipid-telodendrimer hybrid nanoparticles are useful for efficient loading of small-molecule drugs and biomolecules (e.g., peptides and proteins). For example, the presence of the phospholipid bilayers in the stabilized nanodiscs is important for delivery of certain membrane partitioning amphiphilic therapeutics, e.g., amphotericin B and membrane association protein and transmembrane proteins.

Examples of suitable lipids include, but are not limited to, phospholipids (e.g., phosphatidylcholine (PC), phosphatidic acid (PA), phosphatidylethanolamine (PE), phosphatidylglycerol (PG), phosphatidylserine (PS), and phosphatidylinositol (PI), dimyristoyl phosphatidyl choline (DMPC), distearoyl phosphatidyl choline (DSPC), dioleoyl phosphatidyl choline (DOPC), dipalmitoyl phosphatidyl choline (DPPC), dimyristoyl phosphatidyl glycerol (DMPG), distearoyl phosphatidyl glycerol (DSPG), dioleoyl phosphatidyl glycerol (DOPG), dipalmitoyl phosphatidyl glycerol (DPPG), dimyristoyl phosphatidyl serine (DMPS), distearoyl phosphatidyl serine (DSPS), dioleoyl phosphatidyl serine (DOPS), dipalmitoyl phosphatidyl serine (DPPS), dioleoyl phosphatidyl ethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), 1,2-dielaidoyl-sn-glycero-3-phosphoethanolamine (transDOPE), and cardiolipin). Phospholipids can be lysolipids, which contain only one fatty acid moiety bonded to the glycerol subunit via an ester linkage. Lipid extracts, such as egg PC, heart extract, brain extract, liver extract, and soy PC, are also useful in the present disclosure. The lipids can include derivatized lipids, such as PEGylated lipids. Derivatized lipids can include, for example, DSPE-PEG2000, cholesterol-PEG2000, DSPE-polyglycerol, or other derivatives generally known in the art.

Lipidoid-telodendrimer hybrid nanoparticles can comprise a variety of lipidoids. Lipidoids can comprise one or more positively charged groups (e.g., primary amine, secondary amine, tertiary amine, or guanidine groups) and one or more hydrophobic moieties (e.g., single aliphatic or double aliphatic carbon chains). For example, the lipidoid has single aliphatic or double aliphatic carbon chains comprising 2 to 50 carbons, including all integer number of carbons and ranges therebetween. A lipidoid can be an amphiphilic molecule comprising positively charged or negatively charged head groups conjugated with hydrophobic molecules. Examples of suitable lipidoids are commercially available or can be made using methods known in the art.

Without intending to be bound by any particular theory, it is considered that positively charged group(s) of the lipidoids can bind to negatively charged groups on a protein surface based on electrostatic interactions, which can provide efficient protein loading, and offer enhanced cell membrane permeability and even improved endosomal escape for cargo proteins. The hydrophobic lipidoid components can also interact with hydrophobic groups on telodendrimers based on hydrophobic-hydrophobic interactions to produce small (e.g., diameter of 100 nm or less) and stable nanoparticles. Hydrophobic domain(s) in the resultant nanoparticles can interact with the hydrophobic domains in proteins based on hydrophobic-hydrophobic interactions in addition to the above mentioned electrostatic interactions for stable protein conjugation. Moreover, the hydrophobic components in the nanoparticles are also useful to render assistance to intracellular delivery of cargo proteins.

Examples of suitable lipidoids include, but are not limited to, 1,1′-(propane-1,3-diylbis(methylazanediyl))bis(hexadecan-2-ol), 1-((3-(dimethylamino)propyl)amino)hexadecan-2-ol, or 1,1′-((((((2-hydroxyhexadecyl)amino)methyl)azanediyl)bis(ethane-2,1-diyl))bis(oxy))bis(hexadecan-2-ol).

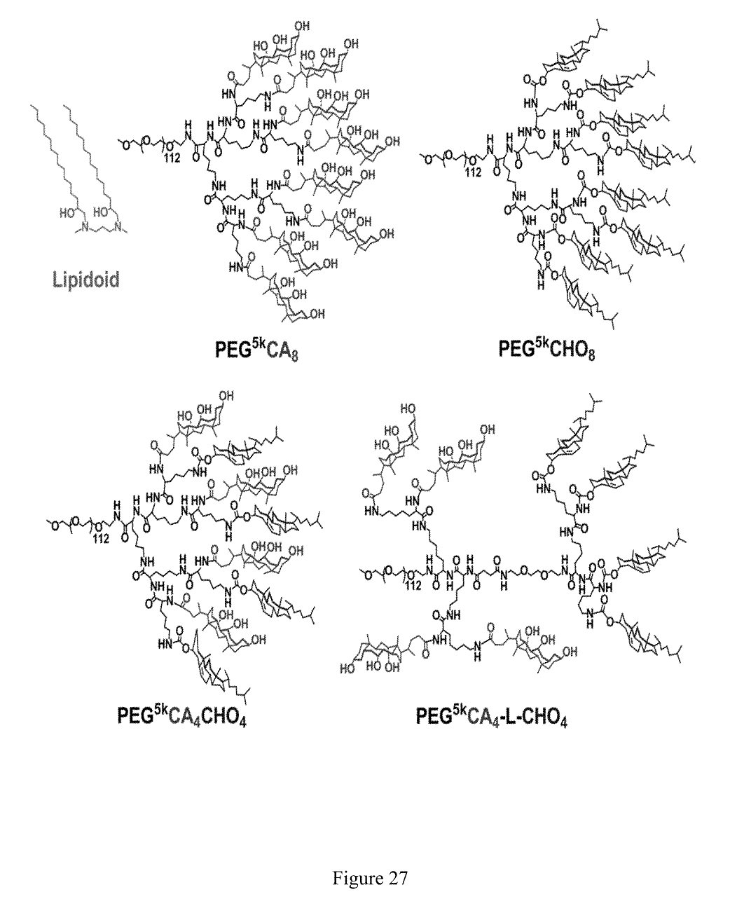

Lipidic compound (lipidic molecule)-telodendrimer hybrid nanoparticles can comprise a variety of telodendrimers. For example, are functional segregated telodendrimers having, for example, two or three functional segments. The telodendrimers can be tri-block telodendrimers with segregated functional regions. Telodendrimers can be referred to as, for example, G1, G2, or G3 telodendrimers. Examples of telodendrimers are shown in FIGS. 27-29.

The telodendrimers may have a PEG groups. Without intending to be bound by any particular theory, it is considered that the PEG layer serves as a stealth hydrophilic shell to stabilize the nanoparticle and to avoid systemic clearance by the reticuloendothelial system (RES); the intermediate layer contains for example, amphiphilic oligo-cholic acid, riboflavin, or chlorogenic acid and can further stabilize nanoparticle and cage drug molecules in the core of nanoparticle; the interior layer contains drug-binding building blocks, such as vitamins (α-tocopherol, riboflavin, folic acid, retinoic acid, etc.) functional lipids (ceramide), chemical extracts (rhein, coumarin, curcumin, etc.) from herbal medicine to increase the affinity to drug molecules.

In various examples, a telodendrimer has a structure of formula (I):

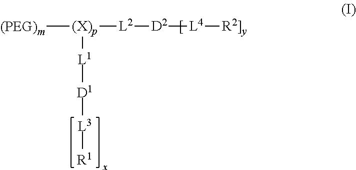

where PEG is optionally present and is a polyethylene glycol moiety, where PEG has a molecular weight of 44 Da to 100 kDa; X is optionally present and is a branched monomer unit; each L¹ is independently optional and is a linker group; each L² is independently optional and is a linker group; each L³ is independently optional and is a linker group; each L⁴ is independently optional and is a linker group; D¹ is optional and is a dendritic polymer moiety having one or more branched monomer units (X); D² is a dendritic polymer having one or more branched monomer units (X), where multiple branched monomers may be linked together by a linking moiety (L⁴); each R¹ is an end group of the dendritic polymer and is independently at each occurrence in the compound selected from the group consisting of cholic acid, cholesterol, rhein, coumarin, curcumin, flavin, isoflavin, riboflavin, retinol, retinoic acid, chlorogenic acid; anthraquinone, xanthenone, Vitamin E, D-α-tocopherol succinate, vitamins, lipids, fatty acids, bile acids, naturally-isolated compound moieties, and drugs; each R² is an end group of the dendritic polymer and is independently at each occurrence in the compound selected from the group consisting of positively or negatively charged groups (e.g., arginine, lysine, guanidine, amine, amidine, tetrazole, hydroxyl, carboxyl, phosphate, sulfonate, methanesulfonamide, sulfonamide, or oxalic acid functional groups), a hydrophobic group, a hydrophilic group, an amphiphilic group, and a drug (R² can comprise two different end groups, where one half of the R² end groups are one of said group and one half of the R² end groups are a second of said group; subscript x is an integer from 1 to 64, subscript y is an integer from 1 to 64, subscript p is an integer from 1 to 32; and subscript m is an integer from 0 to 32.

In an example, at each occurrence in the compound the branched monomer unit (X) in the compound of formula (I) is independently selected from the group consisting of a diamino carboxylic acid moiety, a dihydroxy carboxylic acid moiety, and a hydroxyl amino carboxylic acid moiety.

R² groups are end groups of the dendritic polymer and are independently at each occurrence in the compound selected from the group consisting of cholic acid moiety or derivative or analog thereof, cholesterol moiety or derivative or analog thereof, rhein moiety or derivative or analog thereof, coumarin moiety or derivative or analog thereof, curcumin moiety or derivative or analog thereof, flavin moiety or derivative or analog thereof, isoflavin moiety or derivative or analog thereof, riboflavin moiety or derivative or analog thereof, retinol moiety or derivative or analog thereof, retinoic acid moiety or derivative or analog thereof, chlorogenic acid moiety or derivative or analog thereof; anthraquinone moiety or derivative or analog thereof, xanthenone moiety or derivative or analog thereof, Vitamin E moiety or derivative or analog thereof, and D-α-tocopherol succinate moiety or derivative or analog thereof, vitamins or derivative or analog thereof, lipids or derivative or analog thereof, fatty acids or derivative or analog thereof, bile acids or derivative or analog thereof, naturally-isolated compound moieties or derivative or analog thereof, and drugs or derivative or analog thereof. R² groups may also be positively or negative charged moieties. For example they may be arginine, lysine, guanidine, amine (e.g., secondary, tertiary or quaternary amines). In various examples, subscript y is an integer from 2 to 64, including all integer values and ranges therebetween. In an example, subscript y is equal to the number of end groups on the dendritic polymer. In various examples, at least half the number y of R² groups are each independently selected from the group consisting of coumarin moiety or derivative or analog thereof, curcumin moiety or derivative or analog thereof, flavin moiety or derivative or analog thereof, isoflavin moiety or derivative or analog thereof, riboflavin moiety or derivative or analog thereof, retinol moiety or derivative or analog thereof, retinoic acid moiety or derivative or analog thereof, chlorogenic acid moiety or derivative or analog thereof, anthraquinone moiety or derivative or analog thereof, xanthenone moiety or derivative or analog thereof, Vitamin E moiety or derivative or analog thereof, and D-α-tocopherol succinate moiety or derivative or analog thereof, vitamins or derivative or analog thereof, lipids or derivative or analog thereof, fatty acids or derivative or analog thereof, bile acids or derivative or analog thereof, naturally-isolated compound moieties or derivative or analog thereof, and drugs or derivative or analog thereof.

R¹ groups are end groups of the dendritic polymer and are independently at each occurrence in the compound selected from the group consisting of cholic acid moiety or derivative or analog thereof, cholesterol moiety or derivative or analog thereof, rhein moiety or derivative or analog thereof, coumarin moiety or derivative or analog thereof, curcumin moiety or derivative or analog thereof, flavin moiety or derivative or analog thereof, isoflavin moiety or derivative or analog thereof, riboflavin moiety or derivative or analog thereof, retinol moiety or derivative or analog thereof, retinoic acid moiety or derivative or analog thereof, chlorogenic acid moiety or derivative or analog thereof; anthraquinone moiety or derivative or analog thereof, xanthenone moiety or derivative or analog thereof, Vitamin E moiety or derivative or analog thereof, and D-α-tocopherol succinate moiety or derivative or analog thereof, vitamins or derivative or analog thereof, lipids or derivative or analog thereof, fatty acids or derivative or analog thereof, bile acids or derivative or analog thereof, naturally-isolated compound moieties or derivative or analog thereof, and drugs or derivative or analog thereof.

In various examples, the telodendrimer compound of the present disclosure has the following structure:

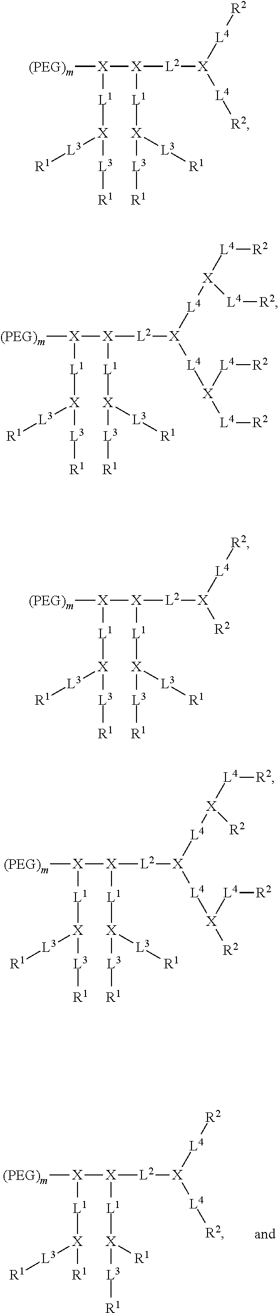

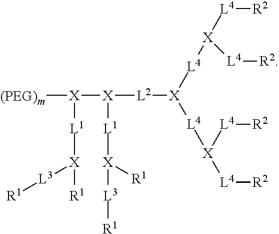

where each branched monomer unit may be a lysine moiety. In these structures, the arm of the telodendrimer comprising the (PEG)ₘ moiety is the hydrophilic segment, the branches of the telodendrimer comprising the L¹ and L³ moieties are the intermediate segments, and the branches of the telodendrimer comprising the L² and L⁴ moieties are the hydrophobic segment. R¹ and R² are as defined herein.

In various examples, at each occurrence in the compound the linker L¹, L², L³ and L⁴ in the compound of formula (I) are independently at each occurrence selected from the group consisting of a polyethylene glycol moiety, polyserine moiety, enzyme cleavable peptide moiety, disulfide bond moiety, acid labile moiety, polyglycine moiety, poly(serine-glycine) moiety, aliphatic amino acid moieties, 6-amino hexanoic acid moiety, 5-amino pentanoic acid moiety, 4-amino butanoic acid moiety, and beta-alanine moiety. In various examples, at each occurrence in the compound the linker L¹, L², and L³ are independently at each occurrence selected from the group consisting of:

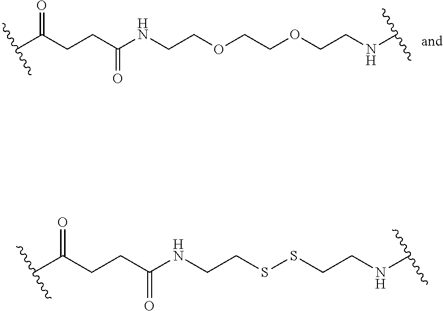 [SMILES: *NCCOCCOCCNC(=O)CCC(*)=O ; *NCCSSCCNC(=O)CCC(*)=O]

in the compound of formula (I). In various examples, the linker L¹, L², L³, L⁴, or a combination thereof comprises a cleavable group in the compound of formula (I). In an example, the cleavable group is a disulfide cleavable moiety in the compound of formula (I).

In various examples, the (PEG)ₘ portion of the compound is selected from the group consisting of:

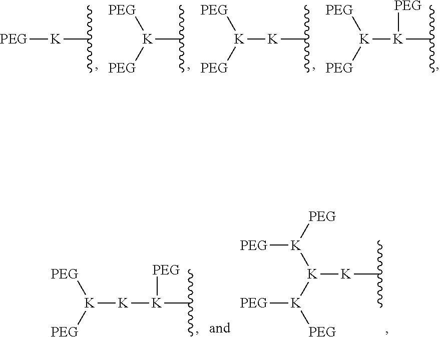

where each K is lysine in the compound of formula (I).

In various examples, each R² is independently selected from a rhein moiety or derivative or analog thereof, cholic acid moiety or derivative or analog thereof, cholesterol moiety or derivative or analog thereof, coumarin moiety or derivative or analog thereof, curcumin moiety or derivative or analog thereof, flavin moiety or derivative or analog thereof, isoflavin moiety or derivative or analog thereof, riboflavin moiety or derivative or analog thereof, retinol moiety or derivative or analog thereof, retinoic acid moiety or derivative or analog thereof, chlorogenic acid moiety or derivative or analog thereof; anthraquinone moiety or derivative or analog thereof, xanthenone moiety or derivative or analog thereof, Vitamin E moiety or derivative or analog thereof, D-α-tocopherol succinate moiety or derivative or analog thereof, vitamins, lipids, fatty acids, bile acids, naturally-isolated compound moieties, and drugs, and combinations thereof in the compound of formula (I).

R¹ is independently at each occurrence in the compound an amphiphilic end group and includes but is not limited to: cholic acid, cholesterol, rhein, coumarin, curcumin, flavin, isoflavin, riboflavin, retinol, retinoic acid, chlorogenic acid; anthraquinone, xanthenone, Vitamin E, D-α-tocopherol succinate, vitamins, lipids, fatty acids, bile acids, naturally-isolated compound moieties, and drugs.

The dendritic polymer of the telodendrimer can be any suitable generation of dendritic polymer, including generation 1, 2, 3, 4, 5, or more, where each “generation” of dendritic polymer refers to the number of branch points encountered between the focal point and the end group following one branch of the dendritic polymer. The dendritic polymer of the telodendrimer can also include partial-generations such as 1.5, 2.5, 3.5, 4.5, 5.5, etc., where a branch point of the dendritic polymer has only a single branch. The various architectures of the dendritic polymer can provide any suitable number of end groups, including, but not limited to, 2 to 128 end groups and all integer value of end groups and ranges therebetween.

The focal point of a dendritic polymer, telodendrimer, dendritic polymer segment, or telodendrimer segment may be any suitable functional group. In some examples, the focal point includes a functional group that allows for attachment of dendritic polymer, telodendrimer, dendritic polymer segment, or telodendrimer segment to another segment. The focal point functional group can be a nucleophilic group including, but not limited to, an alcohol, an amine, a thiol, or a hydrazine. The focal point functional group may also be an electrophile such as an aldehyde, a carboxylic acid, or a carboxylic acid derivative including an acid chloride or an N-hydroxysuccinimidyl ester.

The R¹, R² groups installed at the telodendrimer periphery can be any suitable chemical moiety, including hydrophilic groups, hydrophobic groups, or amphiphilic compounds. Examples of hydrophobic groups include, but are not limited to, long-chain alkanes and fatty acids, fluorocarbons, silicones, certain steroids such as cholesterol, and many polymers including, for example, polystyrene and polyisoprene, polylactide, polyglycolide, and polycaprolactone. Examples of hydrophilic groups include, but are not limited to, alcohols, short-chain carboxylic acids, amines, sulfonates, phosphates, sugars, and certain polymers such as PEG. Examples of amphiphilic compounds include, but are not limited to, molecules that have one or more hydrophilic part and one or more hydrophobic part.

In various examples, each R¹ and R² is independently selected from a rhein moiety or derivative or analog thereof, cholic acid moiety or derivative or analog thereof, coumarin moiety or derivative or analog thereof, curcumin moiety or derivative or analog thereof, flavin moiety or derivative or analog thereof, isoflavin moiety or derivative or analog thereof, retinol moiety or derivative or analog thereof, retinoic acid moiety or derivative or analog thereof, anthraquinone moiety or derivative or analog thereof, xanthenone moiety or derivative or analog thereof, Vitamin E moiety or derivative or analog thereof, D-α-tocopherol succinate moiety or derivative or analog thereof, Vitamins, lipids, fatty acids, Bile acids, naturally-isolated compound moieties, and drugs.

In various examples, each R² is independently selected from a rhein moiety or derivative or analog thereof, cholic acid moiety or derivative or analog thereof, coumarin moiety or derivative or analog thereof, curcumin moiety or derivative or analog thereof, flavin moiety or derivative or analog thereof, isoflavin moiety or derivative or analog thereof, retinol moiety or derivative or analog thereof, retinoic acid moiety or derivative or analog thereof, anthraquinone moiety or derivative or analog thereof, xanthenone moiety or derivative or analog thereof, Vitamin E moiety or derivative or analog thereof, D-α-tocopherol succinate moiety or derivative or analog thereof, vitamins, lipids, fatty acids, bile acids, naturally-isolated compound moieties, and drugs.

In various examples, the end groups of the telodendrimer can alternate between groups. For example, R¹ can be a cholic acid moiety and a rhein moiety and adjacent R¹'s can alternate between these two moieties. This can be applied to R².

In various examples, of lipid-telodendrimer hybrid nanoparticles, the lipid is selected from DMPC, POPC, DPPC, and mixtures thereof, with or without cholesterol, and telodendrimer has R¹ groups comprising amphiphilic moieties, e.g., cholic acid, riboflavin, chlorogenic acid and R² end groups comprising hydrophobic moieties, e.g., rhein, cholesterol, vitamin E and aliphatic chains. For example, with different ratios of lipids and telodendrimers, nanoparticles, e.g., nanodiscs, are formed. For example, with different ratios of lipids and telodendrimers, transmembrane or membrane associated protein/peptide or membrane active small molecular therapeutics, e.g. amphotericin B, form loaded-nanoparticles (e.g., loaded nanodiscs) with a particle size of 100 nm or less.

In various examples of lipidoid-telodendrimer hybrid nanoparticles, the lipidoid is selected from lipidoids comprising two tertiary amine groups and two hydroxyl groups in the polar region and two aliphatic hydrophobic tails and the telodendrimer has one or more or all R¹ end groups comprising amphiphilic moieties, e.g. cholic acid, riboflavin, and chlorogenic acid, and R² end groups comprising hydrophobic moieties, e.g., cholesterol, rhein, vitamin E and aliphatic chains. For example, with different ratios of lipidoid and telodendrimers, the nanoparticles form complexes with protein and/or peptide therapeutics and the resulting nanoparticles (loaded nanoparticles) with a particle size of 100 nm or less.

Examples of telodendrimers are referred to herein using a shorthand description. For example, examples of G1 telodendrimers are referred to using nomenclature such as PEG^{5k}CA₈ in which PEG^{5k} refers to a linear polyethylene glycol moiety with a molecular weight of 5,000 Daltons and CA₈ refers to eight cholic acid(CA) groups (e.g., R¹ and/or R² moieties) conjugated to the dendritic structure. For example, examples of G2 telodendrimers are referred to using nomenclature such as PEG^{5k}CA₄CHO₄ in which PEG^{5k} refers to a linear polyethylene glycol moiety with a molecular weight of 5,000 Daltons and CA₄CHO₄ refers to four cholic acid (CA) groups (e.g., R¹ and/or R² moieties) and four cholesterol (CHO) groups (e.g., R¹ and/or R² moieties) conjugated to alternating peripheral moieties of the dendritic structures, respectively. For example, examples of G3 telodendrimers are referred to using nomenclature such as PEG^{5k}CA₄-L-CHO₄ in which PEG^{5k} refers to a linear polyethylene glycol moiety with a molecular weight of 5,000 Daltons and CA₄ refers that four cholic acid (CA) groups (e.g., R¹ and/or R² moieties) conjugated in the intermediate layer, -L-indicates that a linker molecule is sited between the intermediate layer and the interior layer, and CHO₄ indicates that four cholesterol (CHO) groups (e.g., R¹ and/or R² moieties) are conjugated on the interior dendritic structure.

Telodendrimers of the present disclosure can be synthesized via peptide chemistry, which can control the chemical structure and the architecture of the telodendrimers. Efficient stepwise peptide chemistry allows for reproducibility and scaling up for clinical development. In addition, given their structure, the telodendrimers can self-assemble into micelle nanoparticles with controlled and tunable properties, such as particle size, drug loading capacity and stability. Cholic acid is a facial amphiphilic biomolecule. As a core-forming building block, cholic acid can play a role in stabilizing nanoparticle and the drug molecules loaded in the nanoparticles. Drug-binding bioactive and biocompatible molecules can be introduced into telodendrimer in the core of the micelle to improve the drug loading capacity and stability.

Lipidic compound (lipidic molecule)-telodendrimer hybrid nanoparticles (e.g., lipid (e.g., phospholipid)/lipidoid-telodendrimer hybrid nanoparticles) can have various ratios of lipidic compound (lipidic molecule) (e.g., lipid/lipidoid) to telodendrimer. The ratio of lipidic compound (lipidic molecule) (e.g., lipid/lipidoid) to telodendrimer can be controlled. For example, the ratio of lipidic compound (lipidic molecule) (e.g., lipid/lipidoid) to telodendrimer can be 1:1 to 1:10, including all ratios therebetween (e.g., 1:2, 1:3, 1:4, 1:5, 1:6, 1:7, 1:8, 1:9). In another example, the ratio of lipidic compound (lipidic molecule) (e.g., lipid/lipidoid) to telodendrimer is 1:5.

Lipid-telodendrimer hybrid nanoparticles can be spherical in shape or display as the stabilized bi-layer membranes, e.g. nanodiscs. Lipid-telodendrimer hybrid nanoparticles can comprise one or more small molecule (e.g., small-molecule drug(s)) (small molecule-loaded lipid-telodendrimer hybrid nanoparticles). For example, the hybrid nanoparticle can comprise 1 to 50% (based on hybrid nanoparticle weight) small molecule, including all integer % values and ranges therebetween. The small molecules can be homogenously dispersed within the hybrid nanoparticles or small molecules can preferably partition in the lipid-rich section of nanoparticle.

A small molecule drug is an agent capable of treating and/or ameliorating a condition or disease. The small molecule drugs of the present disclosure also include prodrug forms and drug-like compounds.

Small molecule drugs for small molecule-loaded lipid-telodendrimer hybrid nanoparticles can be surface active drugs. Examples of suitable surface active drugs include, but are not limited to, antibiotics (e.g., dextropropoxyphene, actinomycin D, penicillin G, streptomycin, and sodium fusidate), anticholinergics (e.g., adiphenine, chlorphenoxamine, orphenadrine, penthianate methobromide, and piperidolate), antifungal polyenes (e.g., amphotericin B and nystatin), antihistamines (e.g., bromodiphenylhydramine, chlorcyclizine, diphenhydramine, diphenylpyraline, thenyldiamine, and tripelennamine), antihypertensives (e.g., acetobutolol, oxprenolol, propranolol, thiopental, dibucaine, stadacaine, tetracaine, phenothiazines chlorpromazine, promazine, promethazine, thioridazine, trifluoperazine, and trifluopromazine), thioxanthene tranquilizers (e.g., flupenthixol), tricyclic antidepressants (amitriptyline, butriptyline, clomipramine, desipramine, imipramine, and nortriptyline), doxorubicin, daunorubicin, idarubicin, and the like. One of skill in the art will appreciate that other drugs can be used in the present disclosure.

Lipid-telodendrimer hybrid nanoparticles can comprise one or more peptide (peptide-loaded lipid-telodendrimer hybrid nanoparticles). For example, the hybrid nanoparticle can comprise 1 to 50% (based on hybrid nanoparticle weight) peptide, including all integer % values and ranges therebetween. Peptides can be a fragment of a protein, or synthetic polypeptides. For example, the peptides have 2 to 50 amino acid residues, including all integer number or amino acid residues and ranges therebetween. The peptides(s) can be homogenously dispersed within the hybrid nanoparticles or peptides can preferably partition in the lipid-rich section of nanoparticle.

Examples of suitable peptides for peptide-loaded lipid-telodendrimer hybrid nanoparticles include, but are not limited to, antimicrobial peptides such as, for example, linear (magainin, pardaxin, cecropin, dermaseptin) or cyclic (alamethicin) K-helices, or L-sheets. L-Sheet-forming peptides are cyclized by one (brevinin-1) or more (protegrin I, tachyplesin I, L-defensin-1) disulfide bonds or by lactone formation (gramicidin S, tyrocidin).

Lipid-telodendrimer hybrid nanoparticles can comprise one or more proteins (e.g., transmembrane proteins) (protein-loaded lipid-telodendrimer hybrid nanoparticles). For example, the hybrid nanoparticle can comprise 1 to 50% (based on hybrid nanoparticle weight) protein, including all integer % values and ranges therebetween. The proteins(s) can be homogenously dispersed within the hybrid nanoparticles or proteins can preferably partition in the lipid-rich section of nanoparticle.

Examples of suitable proteins for protein-loaded lipid-telodendrimer hybrid nanoparticles include, but are not limited to, transmembrane proteins such as, for example, light absorption-driven transporters (e.g., rhodopsin), oxidoreduction-driven transporters (e.g., coenzyme Q-cytochrome c reductase), electrochemical potential-driven transporters (e.g., V-ATPases), P—P-bond hydrolysis-driven transporters (e.g., p-type calcium ATPase, ABC transporter), alpha-helical channels including ion channels (e.g., voltage-gated ion channel like protein), transmembrane enzymes (e.g., methane monoxygenases, Rhomboid protease), proteins with alpha-helical transmembrane anchors (e.g., Cytochrome P450 oxidases), β-barrels composed of a single polypeptide chain (e.g., outer membrane protein G porin family proteins), and β-barrels composed of a plurality of polypeptide chains (e.g., outer membrane efflux proteins).

In the case of lipid-telodendrimer hybrid nanoparticles comprising one or more small molecule (e.g., small-molecule drug(s)) or peptides, it is considered that the lipid(s) form a bilayer structure and the telodendrimer(s) at least partially encapsulate (e.g., completely encapsulate) the lipid bilayer structure and small molecule(s)/peptide(s) is/are sequestered (e.g., intercalated or at least partially intercalated) in the lipid bilayer structure.

Lipidoid-telodendrimer hybrid nanoparticles can be spherical in shape. Lipidoid-telodendrimer hybrid nanoparticles can comprise one or more protein (protein-loaded lipidoid-telodendrimer hybrid nanoparticles). For example, the hybrid nanoparticle can comprise 1 to 50% (based on hybrid nanoparticle weight) protein, including all integer % values and ranges therebetween. The protein(s) can be homogenously dispersed within the hybrid nanoparticles, or small molecules can preferably partition in the lipid-rich section of nanoparticle.

Examples of suitable proteins for protein-loaded lipidoid-telodendrimer hybrid nanoparticles include, but are not limited to, nucleoproteins, glycoproteins, lipoproteins, immunotherapeutic proteins, porcine somatotropin for increasing feed conversion efficiency in a pig, insulin, growth hormone, buserelin, leuprolide, interferon, gonadorelin, calcitonin, cyclosporin, lisinopril, captopril, delapril, tissue plasminogen activator, epidermal growth factor, fibroblast growth factor (acidic or basic), platelet derived growth factor, transforming growth factor (alpha or beta), vasoactive intestinal peptide, tumor necrosis factor; hormones such as glucagon, calcitronin, adrecosticotrophic hormone, follicle stimulating hormone, enkaphalins, β-endorphin, somatostin, gonado trophine, α-melanocyte stimulating hormone. Additional examples include bombesin, atrial naturiuretic peptides and luteinizing hormone releasing (LHRH), substance P, vasopressins, α-globulins, transferrins, fibrinogens, lipoproteins, β-globulins, prothrombin (bovine), ceruloplasmin, α₂-glycoproteins, α₂-globu\ddot{ν}ns, fetuin (bovine), -lipoproteins, α, -globulins, albumin and prealbumin, bovine serum albumin, green fluorescent protein, diphtheria toxins, lysozyme, trypsin, cytochrome c, saporin, ribonuclease A, IgG, and antibodies.

Lipidoid-telodendrimer hybrid nanoparticles can comprise one or more peptide (peptide-loaded lipidoid-telodendrimer hybrid nanoparticles). For example, the hybrid nanoparticle can comprise 1 to 50% (based on hybrid nanoparticle weight) peptide, including all integer % values and ranges therebetween. Peptides can be a fragment of a protein, or synthetic polypeptides. For example, the peptides have 2 to 50 amino acid residues, including all integer number or amino acid residues and ranges therebetween. The protein(s) can be homogenously dispersed within the hybrid nanoparticles, or small molecules can preferably partition in the lipid-rich section of nanoparticle.

Examples of suitable peptides for peptide-loaded lipidoid-telodendrimer hybrid nanoparticles include, but are not limited to, antimicrobial peptides such as, for example, linear (magainin, pardaxin, cecropin, dermaseptin) or cyclic (alamethicin) K-helices, or L-sheets. L-Sheet-forming peptides are cyclized by one (brevinin-1) or more (protegrin I, tachyplesin I, L-defensin-1) disulfide bonds or by lactone formation (gramicidin S, tyrocidin).

Additional examples of suitable peptides for peptide-loaded lipidoid-telodendrimer hybrid nanoparticles include, but are not limited to, antimicrobial peptides such as, for example, linear (magainin, pardaxin, cecropin, dermaseptin) or cyclic (alamethicin) K-helices, or L-sheets. L-Sheet-forming peptides are cyclized by one (brevinin-1) or more (protegrin I, tachyplesin I, L-defensin-1) disulfide bonds or by lactone formation (gramicidin S, tyrocidin).

Lipidic compound (lipidic molecule)-telodendrimer hybrid nanoparticles or small molecule/peptide/protein-loaded lipidic compound (lipidic molecule)-telodendrimer hybrid nanoparticles (e.g., lipid/lipidoid-telodendrimer hybrid nanoparticles (or protein/peptide loaded hybrid nanoparticles or small molecule-loaded hybrid nanoparticles)) can have a variety of sizes. Typically, an increase in lipidic compound (lipidic molecule) (e.g., lipid or lipidoid) content results in an increase in the size of the hybrid nanoparticle. It may be desirable that the hybrid nanoparticles or loaded hybrid nanoparticles have a size (i.e., longest dimension) of 100 nm or less. For example, the hybrid nanoparticles or loaded hybrid nanoparticles have a size (e.g., an average particle size) of 1 nm to 100 nm, including all integer nm values and ranges therebetween. In various examples, the hybrid nanoparticles or loaded hybrid nanoparticles have a size (e.g., an average particle size) of 10 nm to 90 nm, 10 nm to 80 nm, 10 nm to 70 nm, or 10 nm to 60 nm. For example, the particle size is a hydrodynamic size. Particle size can be determined by methods known in the art. For example, the particle size is determined by dynamic light scattering.

Lipidic compound (lipidic molecule)-telodendrimer hybrid nanoparticles or small molecule/peptide/protein-loaded lipidic compound (lipidic molecule)-telodendrimer hybrid nanoparticles (e.g., lipid/lipidoid-telodendrimer hybrid nanoparticles (or protein/peptide loaded hybrid nanoparticles or small molecule-loaded hybrid nanoparticles)) can have various morphology. For example, the hybrid nanoparticles have spherical or nanodisc morphology.

Lipidic compound (lipidic molecule)-telodendrimer hybrid nanoparticles (e.g., lipid/lipidoid-telodendrimer hybrid nanoparticles) comprising a protein and/or peptide and/or small-molecule (protein/peptide-loaded hybrid nanoparticles or small molecule-loaded hybrid nanoparticles) can exhibit desirable stability. For example, an aqueous suspension of lipid-telodendrimer hybrid nanoparticles comprising a small-molecule (e.g., a small-molecule drug) do not exhibit observable precipitation (i.e., free small molecule) and/or no change in size (e.g., no aggregate formation) for at least 24 hours after production of the lipid-telodendrimer hybrid nanoparticles comprising the small-molecule. In various examples, an aqueous suspension of lipid-telodendrimer hybrid nanoparticles comprising a small-molecule (e.g., a small-molecule drug) do not exhibit observable precipitation (i.e., free small molecule) and/or no change in size (e.g., no aggregate formation) for at least 5, 10, 15, 20, or 25 days after production of the lipid/lipidoid-telodendrimer hybrid nanoparticles comprising the small-molecule.

A composition can comprise one or more lipidic compound (lipidic molecule)-telodendrimer hybrid nanoparticles or small molecule/peptide/protein-loaded lipidic compound (lipidic molecule)-telodendrimer hybrid nanoparticles (e.g., lipid/lipidoid-telodendrimer hybrid nanoparticles or protein/peptide loaded hybrid nanoparticles or small molecule-loaded hybrid nanoparticles). The composition can comprise hybrid nanoparticles having the same or different composition. For example, the composition is an aqueous solution comprising one or more lipidic compound (lipidic molecule)-telodendrimer hybrid nanoparticles or small molecule/peptide/protein-loaded lipidic compound (lipidic molecule)-telodendrimer hybrid nanoparticles (e.g., lipid/lipidoid-telodendrimer hybrid nanoparticles or protein/peptide loaded hybrid nanoparticles or small molecule-loaded hybrid nanoparticles).

The hybrid nanoparticles of the present disclosure can be formulated in a variety of different manners to provide various compositions. For example, a composition comprises one or more hybrid nanoparticles of the present disclosure and a pharmaceutically acceptable carrier. Pharmaceutically acceptable carriers are determined in part by the particular composition being administered, as well as by the particular method used to administer the composition. Accordingly, there are a wide variety of suitable formulations of pharmaceutical compositions of the present disclosure (see, e.g., Remington's Pharmaceutical Sciences, 20^{th} ed., 2003, supra). Effective formulations include oral and nasal formulations, formulations for parenteral administration, and compositions formulated for with extended release.

Formulations suitable for oral administration can consist of (a) liquid solutions, such as an effective amount of a compound of the present disclosure suspended in diluents, such as water, saline or PEG 400; (b) capsules, sachets, depots or tablets, each containing a predetermined amount of the active ingredient, as liquids, solids, granules or gelatin; (c) suspensions in an appropriate liquid; (d) suitable emulsions; and (e) patches. The liquid solutions described above can be sterile solutions. The pharmaceutical forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The composition can, if desired, also contain other compatible therapeutic agents. Preferred pharmaceutical preparations can deliver the compounds of the disclosure in a sustained release formulation.

Pharmaceutical preparations useful in the present disclosure also include extended-release formulations. In some examples, extended-release formulations useful in the present disclosure are described in U.S. Pat. No. 6,699,508, which can be prepared according to U.S. Pat. No. 7,125,567, both patents incorporated herein by reference.

The pharmaceutical preparations are typically delivered to a mammal, including humans and non-human mammals. Non-human mammals treated using the present methods include domesticated animals (i.e., canine, feline, murine, rodentia, and lagomorpha) and agricultural animals (bovine, equine, ovine, porcine).

In practicing the methods of the present disclosure, the pharmaceutical compositions can be used alone, or in combination with other therapeutic or diagnostic agents.

In an aspect, the present disclosure provides methods of making lipidic compound (lipidic molecule)-telodendrimer hybrid nanoparticles (e.g., lipid/lipidoid-telodendrimer hybrid nanoparticles). The hybrid nanoparticles can be made using methods described herein. Also provided are methods of making protein/peptide or small-molecule loaded hybrid nanoparticles. The protein/peptide or small-molecule loaded hybrid nanoparticles can be made using methods described herein.

For example, lipidoid-telodendrimer hybrid nanoparticles can be formed by mixing lipidoid(s) and/or telodendrimer(s) at desired mass ratios in an aqueous ethanol solution comprising citrate ions (e.g., a solution of 90% ethanol and 10% 10 mM sodium citrate (by volume)) resulting in formation of lipidoid-telodendrimer nanoparticles. The resulting solution of lipidoid-telodendrimer nanoparticles can be diluted with phosphate buffered saline (PBS, 1×) (e.g., 10 times volume of PBS), and dialyzed (e.g., against PBS (1×) for 4 h). The lipidoid-telodendrimer nanoparticle solution was then mixed with protein(s) and or peptide(s) at a desired ratio (protein/nanoparticle, w/w) to form a hybrid nanoparticle-protein/peptide complex. The complex solution can be stored in sealed vessels at 4° C.

For example, lipid-telodendrimer hybrid nanoparticles or small-molecule loaded can be made using thin film hydration methods. In an example, lipid(s), telodendrimer(s), and, optionally, small molecules (e.g., small-molecule drugs) were dissolved in methanol/CHCl₃ (1/1, v/v) solution with triethylamine, and the organic solvents removed under vacuum to form a thin film of mixture of lipid(s), telodendrimer(s), and, optionally small molecules. The thin film was then hydrated (e.g., in PBS) to form the lipid-telodendrimer nanoparticles without/with small molecules loaded.

In an aspect, the present disclosure provides methods of using lipidic compound (lipidic molecule)-telodendrimer hybrid nanoparticles (e.g., lipid/lipidoid-telodendrimer hybrid nanoparticles). For example, lipid-telodendrimer hybrid nanoparticles comprising one or more small molecules or lipidoid-telodendrimer hybrid nanoparticles comprising one or more protein(s) and/or peptide(s) can be used in methods of small-molecule or protein/peptide delivery.

The lipidic compound (lipidic molecule)-telodendrimer hybrid nanoparticles (e.g., lipid/lipidoid-telodendrimer hybrid nanoparticles) of the present disclosure can be used to treat any disease requiring the administration of a drug, such as by sequestering a hydrophobic drug in the interior of a hybrid nanoparticle or by stabilizing a protein with one or more hybrid nanoparticle.

For example, the present disclosure provides a method of treating a disease, including administering to an individual in need of such treatment a therapeutically effective amount of a hybrid nanoparticle of the present disclosure, where the hybrid nanoparticle comprises a drug or peptide. In some examples, the drug is a hydrophobic drug sequestered in the interior of a nanoparticle. In some examples, a nanoparticle also includes an imaging agent. The imaging agent can be a covalently attached to a conjugate of a nanoparticle, or the imaging agent can be sequestered in the interior of a nanoparticle. In some other examples, both a hydrophobic drug and an imaging agent are sequestered in the interior of a nanoparticle. In still other examples, both a drug and an imaging agent are covalently linked to a conjugate or conjugates of a nanoparticle. In yet other examples, the nanoparticle can also include a radionuclide.

Hybrid nanoparticles of the present disclosure can be administered to an individual for treatment, e.g., of hyperproliferative disorders including cancer such as, but not limited to: carcinomas, gliomas, mesotheliomas, melanomas, lymphomas, leukemias, adenocarcinomas, breast cancer, ovarian cancer, cervical cancer, glioblastoma, leukemia, lymphoma, prostate cancer, and Burkitt's lymphoma, head and neck cancer, colon cancer, colorectal cancer, non-small cell lung cancer, small cell lung cancer, cancer of the esophagus, stomach cancer, pancreatic cancer, hepatobiliary cancer, cancer of the gallbladder, cancer of the small intestine, rectal cancer, kidney cancer, bladder cancer, prostate cancer, penile cancer, urethral cancer, testicular cancer, cervical cancer, vaginal cancer, uterine cancer, ovarian cancer, thyroid cancer, parathyroid cancer, adrenal cancer, pancreatic endocrine cancer, carcinoid cancer, bone cancer, skin cancer, retinoblastomas, multiple myelomas, Hodgkin's lymphoma, and non-Hodgkin's lymphoma (see, e.g., CANCER: PRINCIPLES AND PRACTICE (DeVita, V. T. et al. eds 2008) for additional cancers).

Other diseases that can be treated by hybrid nanoparticle of the present disclosure include: (1) inflammatory or allergic diseases such as systemic anaphylaxis or hypersensitivity responses, drug allergies, insect sting allergies; inflammatory bowel diseases, such as Crohn's disease, ulcerative colitis, ileitis and enteritis; vaginitis; psoriasis and inflammatory dermatoses such as dermatitis, eczema, atopic dermatitis, allergic contact dermatitis, urticaria; vasculitis; spondyloarthropathies; scleroderma; respiratory allergic diseases such as asthma, allergic rhinitis, hypersensitivity lung diseases, and the like, (2) autoimmune diseases, such as arthritis (rheumatoid and psoriatic), osteoarthritis, multiple sclerosis, systemic lupus erythematosus, diabetes mellitus, glomerulonephritis, and the like, (3) graft rejection (including allograft rejection and graft-v-host disease), and (4) other diseases in which undesired inflammatory responses are to be inhibited (e.g., atherosclerosis, myositis, neurological conditions such as stroke and closed-head injuries, neurodegenerative diseases, Alzheimer's disease, encephalitis, meningitis, osteoporosis, gout, hepatitis, nephritis, sepsis, sarcoidosis, conjunctivitis, otitis, chronic obstructive pulmonary disease, sinusitis and Behcet's syndrome).

In addition, the hybrid nanoparticle of the present disclosure are useful for the treatment of infection by pathogens such as viruses, bacteria, fungi, and parasites. Other diseases can be treated using the hybrid nanoparticles of the present disclosure.

The hybrid nanoparticle of the present disclosure can be administered as frequently as necessary, including hourly, daily, weekly or monthly. The compounds utilized in the pharmaceutical method of the disclosure are administered at the initial dosage of about 0.0001 mg/kg to about 1000 mg/kg daily. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied depending upon the requirements of the patient, the severity of the condition being treated, and the compound being employed. For example, dosages can be empirically determined considering the type and stage of disease diagnosed in a particular patient. The dose administered to a patient, in the context of the present disclosure should be sufficient to effect a beneficial therapeutic response in the patient over time. The size of the dose also will be determined by the existence, nature, and extent of any adverse side-effects that accompany the administration of a particular compound in a particular patient. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. For convenience, the total daily dosage may be divided and administered in portions during the day, if desired. Doses can be given daily, or on alternate days, as determined by the treating physician. Doses can also be given on a regular or continuous basis over longer periods of time (weeks, months or years), such as through the use of a subdermal capsule, sachet or depot, or via a patch or pump.

The pharmaceutical compositions can be administered to the patient in a variety of ways, including topically, parenterally, intravenously, intradermally, subcutaneously (e.g., subcutaneous injection), intramuscularly, intratumorally (e.g., intratumoral injection), intercranially (e.g., intercranial infusion), colonically, intraperitoneally (e.g., rectally), orally, or nasally, such as via inhalation.

In practicing the methods of the present disclosure, the pharmaceutical compositions can be used alone, or in combination with other therapeutic or diagnostic agents. The additional drugs used in the combination protocols of the present disclosure can be administered separately or one or more of the drugs used in the combination protocols can be administered together, such as in an admixture. Where one or more drugs are administered separately, the timing and schedule of administration of each drug can vary. The other therapeutic or diagnostic agents can be administered at the same time as the compounds of the present disclosure, separately or at different times.

The steps of the method described in the various embodiments and examples disclosed herein are sufficient to carry out the methods of the present disclosure. Thus, in an embodiment, a method consists essentially of a combination of the steps of one or more of the methods disclosed herein. In another embodiment, a method consists of such steps.

The following Statements provide various non-limiting examples of lipidic compound (lipidic molecule)-telodendrimer hybrid nanoparticles of the present disclosure, compositions comprising one or more hybrid nanoparticles of the present disclosure and methods of using the hybrid nanoparticles or the compositions:

Statement 1. A lipidic compound (lipidic molecule)-telodendrimer hybrid nanoparticle (e.g., lipid/lipidoid-telodendrimer hybrid nanoparticle) comprising: a lipidic compound (lipidic molecule) (e.g., lipid or lipidoid); a telodendrimer. In various examples, the hybrid nanoparticle has a size of 1 nm to 100 nm.
Statement 2. A lipidic compound (lipidic molecule)-telodendrimer hybrid nanoparticle (e.g., lipid/lipidoid-telodendrimer hybrid nanoparticle) according to Statement 1, where the hybrid nanoparticle comprises a lipid and further comprises a small molecule (e.g., a small-molecule drug) and/or a peptide and/or a protein (e.g., transmembrane protein).
Statement 3. A lipidic compound (lipidic molecule)-telodendrimer hybrid nanoparticle (e.g., lipid/lipidoid-telodendrimer hybrid nanoparticle) according to Statement 1, where the hybrid nanoparticle comprises a lipidoid and further comprises a protein and/or peptide.
Statement 4. A lipidic compound (lipidic molecule)-telodendrimer hybrid nanoparticle (e.g., lipid/lipidoid-telodendrimer hybrid nanoparticle) according to any one of Statements 1-3, where the telodendrimer is a functional segregated telodendrimer of the present disclosure. In various examples, the telodendrimer comprises one, two, or three functional segments and/or one or more poly(ethylene glycol) (PEG) groups/moieties.
Statement 5. A lipidic compound (lipidic molecule)-telodendrimer hybrid nanoparticle (e.g., lipid/lipidoid-telodendrimer hybrid nanoparticle) according to any one of the preceding Statements, where the lipidic compound-telodendrimer hybrid nanoparticle further comprises cholesterol.
Statement 6. A composition comprising one or more lipidic compound (lipidic molecule)-telodendrimer hybrid nanoparticle (e.g., lipid/lipidoid-telodendrimer hybrid nanoparticle) of the present disclosure (e.g., one or more lipidic compound (lipidic molecule)-telodendrimer hybrid nanoparticle of any one of the preceding claims).
Statement 7. A composition according to Statement 6, where the composition comprises one or more lipid-telodendrimer hybrid nanoparticle, one or more lipidoid-telodendrimer hybrid nanoparticle, or a combination thereof.
Statement 8. A composition according to Statement 6 or 7, where one or more of the one or more lipidic compound-telodendrimer hybrid nanoparticle further comprises a small molecule and/or a peptide and/or a protein.
Statement 9. A composition according to any one of Statements 6-8, where one or more of the one or more lipidic compound-telodendrimer hybrid nanoparticle further comprises cholesterol.
Statement 10. A composition according to any one of Statements 6-9, where the composition is an aqueous composition (e.g., the composition further comprises an aqueous component such as, for example, water or saline).
Statement 11. A composition according to any one of Statements 6-9, where the composition is a solid.
Statement 12. A composition according to any one of Statements 6-11, where the composition further comprises one or more pharmaceutically acceptable carrier.
Statement 13. A method of delivering a small molecule (e.g., small-molecule drug) and/or protein and/or peptide comprising: administering a plurality of lipidic compound (lipidic molecule)-telodendrimer hybrid nanoparticles (e.g., lipid/lipidoid-telodendrimer hybrid nanoparticles) of the present disclosure (e.g., a plurality of lipidic compound (lipidic molecule)-telodendrimer hybrid nanoparticles of any one of Statements 1-5) or a composition of the present disclosure (e.g., a composition of any one of Statements 6-12) to an individual. Statement 14. A method of delivering a small molecule (e.g., small-molecule drug) and/or protein and/or peptide according to Statement 13, where the lipidic compound is a lipid, lipidoid, or a combination thereof.
Statement 15. A method of delivering a small molecule (e.g., small-molecule drug) and/or protein and/or peptide according to Statement 13 or 14, where the one or more of the lipidic compound-telodendrimer hybrid nanoparticles or one or more of the lipidic compound-telodendrimer hybrid nanoparticles of the composition comprise cholesterol.
Statement 16. A method of delivering a small molecule (e.g., small-molecule drug) and/or protein and/or peptide according to any one of Statements 13-15, where the individual is a mammal (e.g., a human mammal or non-human mammal).
Statement 17. A method of delivering a small molecule (e.g., small-molecule drug) and/or protein and/or peptide according to any one of Statements 13-16, where the administration is topical, parenteral, intravenous, intradermal, subcutaneous, intramuscular, intratumoral, intercranial, colonical, intraperitoneal, oral, or nasal.

The following examples further describe the disclosure. These examples are intended to be illustrative and not limiting in any way.

### Example

The following describes the preparation and characterization of examples of lipid-telodendrimer nanoparticles of the present disclosure. The following also describes an example of the use of examples the nanoparticles for drug delivery.

Nanoparticle-mediated intracellular delivery of protein therapeutics provides opportunities to treat, for example, brain cancers via intracranial administration to enhance tumor cellular uptake and prevent the leakage of cytotoxic proteins, e.g. diphtheria toxin, into systemic circulation. A facile strategy to precisely engineer lipid-like nanoparticles offers enhanced cell membrane permeability of proteins to reach the intracellular targets. For example, co-assembly of lipidoid (or lipid) and telodendrimers with a three-layered architecture, a linear-dendritic hybrid block copolymer, is able to fine-tune the properties of nanoparticles. The optimized shape, structure and ratio of telodendrimers in the systems effectively prevent aggregation of lipidoids (or lipids), and minimize their surface charge potential accompanying with reduced hemolytic activity and cytotoxicity of cationic lipidoids, yielding neutral, well-dispersed sub-100 nm lipidoid-telodendrimer or lipid-telodendrimer hybrid nanoparticles. The lipidoid-telodendrimer nanoparticles can potently deliver cytotoxins or small molecules into intracranial human glioblastoma multiforme tumor cells with the assistance of convection-enhanced delivery, affording the suppression of tumor growth. The lipid-telodendrimer hybrid nanodiscs can be used for loading of antifungal drugs such as amphotericin B. This study presents a novel strategy for the construction of lipidoid (or lipid)-telodendrimer hybrid nanoparticles for the delivery of proteins and drugs for cancer treatment and antifungal applications.

To meet the demand of CED (“convective-enhanced delivery”) of proteins to intracellular sites for efficient brain tumor treatment, the ideal delivery vehicles are generally required to have: (i) sub-100 nm particle sizes to facilitate the fusion of nanotherapeutics in brain extracellular matrix, (ii) neutral or negative surface charges to reduce non-specific binding to negatively-charged plasma membrane in the brain parenchyma, (iii) slightly viscous surface materials such as polyethylene glycol (PEG) to reduce backflow and to reduce binding to brain cells, (iv) low hemolytic activity and low delivery vehicle-related cytotoxicity to avoid hemolysis and inflammation, (v) high loading capacity and high delivery efficiency to guarantee efficient intracellular protein delivery, (vi) reliable conjugation of proteins to prevent protein denaturation. To create delivery vehicles according to these requirements, we present herein a novel strategy of precisely tunable engineering of lipidoid-telodendrimer hybrid NPs for intracellular protein delivery in brain tumor treatment.

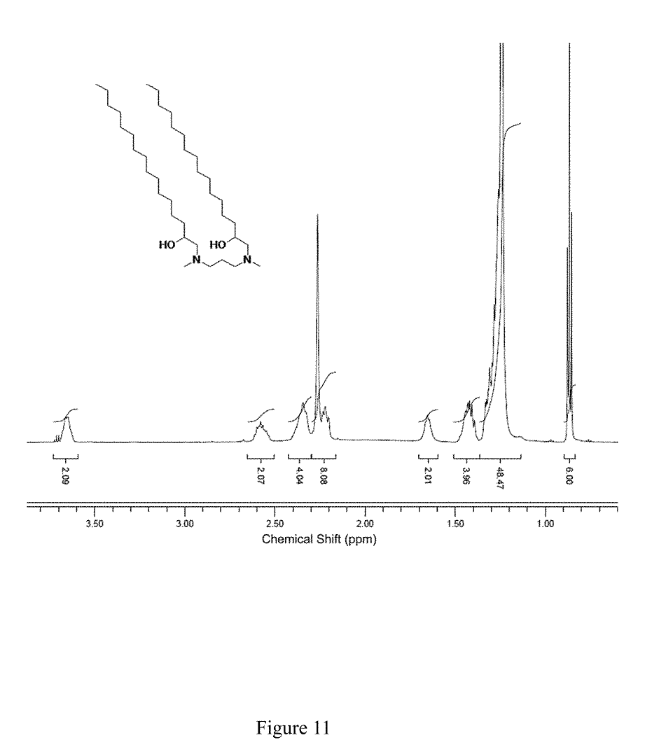

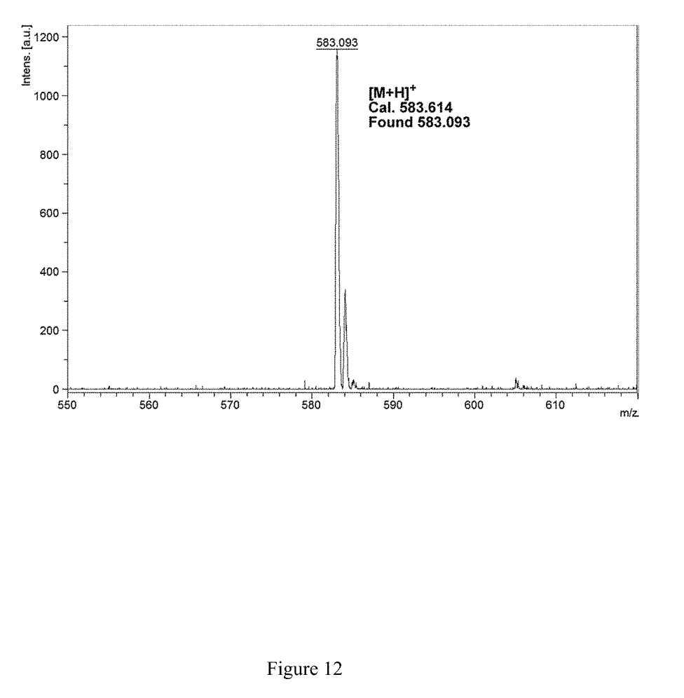

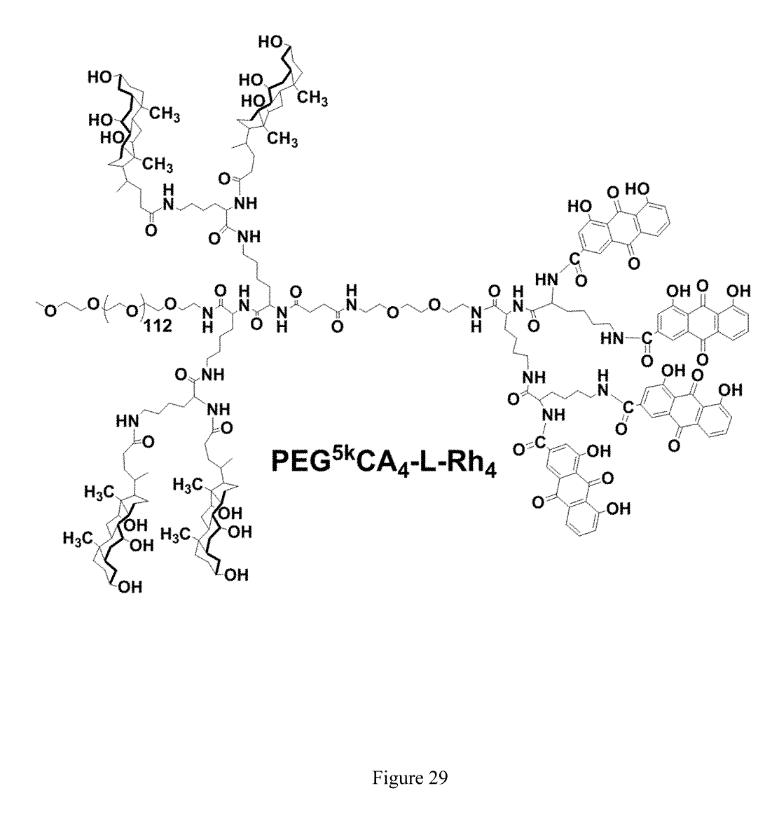

Cationic lipid-like materials, lipidoids, exhibit the ability of efficient intracellular delivery of genes and proteins in vitro and in vivo. A cationic lipidoid, made from 1,2-epoxyhexadecane and N,N′-dimethyl-1,3-propanediamine (FIGS. 11, 12, and 27), has been identified through a combinatorial strategy for efficient protein delivery. It showed promise for systemic delivery of cytotoxic proteins to treat breast cancer. However, this lipidoid forms liposome-like NPs with a large particle size of ˜129 nm and positive charges (ζ-potential: +8 mV). It has been demonstrated that the smaller particle sizes and neutral or slightly negative particle sizes are preferred for intratumoral drug delivery. Precise control on size, structure and property of the lipidoid NPs that is also critical for CED, however, remains a challenge due to their aggregate nature (FIG. 29) and cationic property. The amphiphilic linear-dendritic copolymer systems (named as telodendrimers) allows for rational design of macromolecular architecture and composition. The rationally designed three-layered telodendrimers provide a versatile platform to interact with lipid nanoparticles via the interior hydrophobic moieties and stabilize the nanocomplex via the intermediate layer of amphiphilic oligo cholic acids together with the hydrophilic PEG. Such telodendrimers offer unique capability to tune the cationic lipidoid system over conventional methods, yielding lipidoid-telodendrimer hybrid NPs with small particle sizes and a close to neutral surface chemistry for CED of proteins to enhance brain tumor retention and cellular uptake of therapeutic proteins.

The lipid-polymer hybrid nanoparticles, that exhibit complementary characteristics of both polymeric nanoparticles and liposomes, are also a new generation of therapeutic delivery platform for small molecule drugs. Many polymers such as poly(lactide-co-glycolide) (PLGA), maltodextrin and polystyrene, and lipids such as PEG-distearoylphosphatidylethanolamine (PEG-DSPE) and 1,2-dipalmitoyl-3-trimethylammonium-propane (DPTAP), were employed to build lipid-polymer hybrid nanoparticles for the delivery of various drugs, including doxorubicin, paclitaxel and docetaxel. We applied versions of telodendrimer systems with PEG-b-dendritic structure to stabilize phospholipid bilayer membranes successfully. However, the large particle sizes and moderate amphotericin B loading efficiency were obtained due to the suboptimal architecture of telodendrimers. With this novel three layered architecture, telodendrimer can efficiently stabilize the edge of phospholipid bilayer membrane to form nanodiscs with sub-60 nm in particle sizes for efficient amphotericin B delivery. The interior moiety of telodendrimer can be engineered to optimize the interactions between telodendrimer and the hydrophobic tail and polar charged head of phospholipid at the edge of the bilayer membrane. We applied the nanodiscs for membrane protein stabilization, which is expected to be provide a powerful tool for both basic research and biomedical detections.

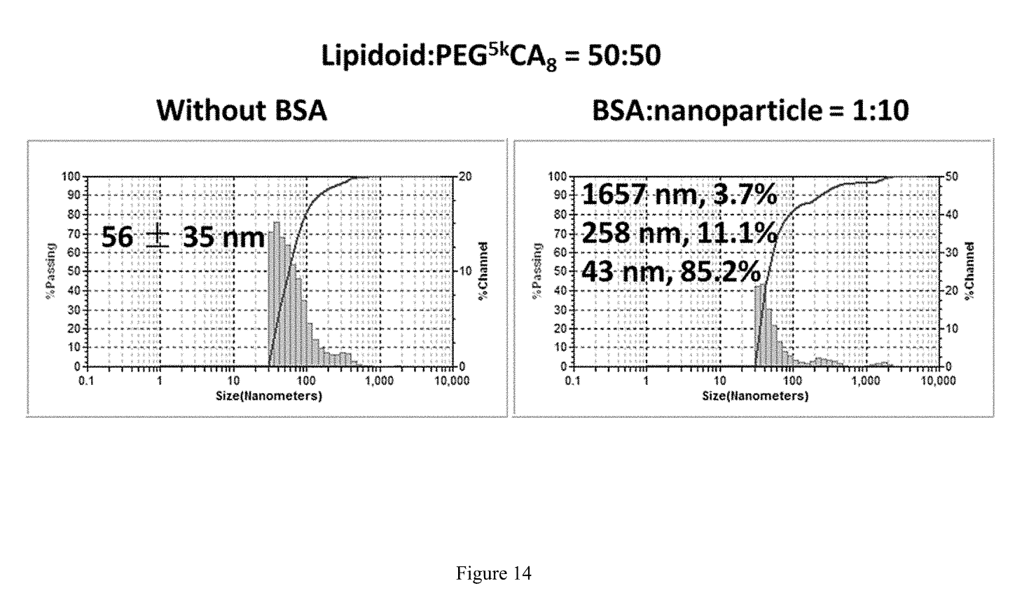

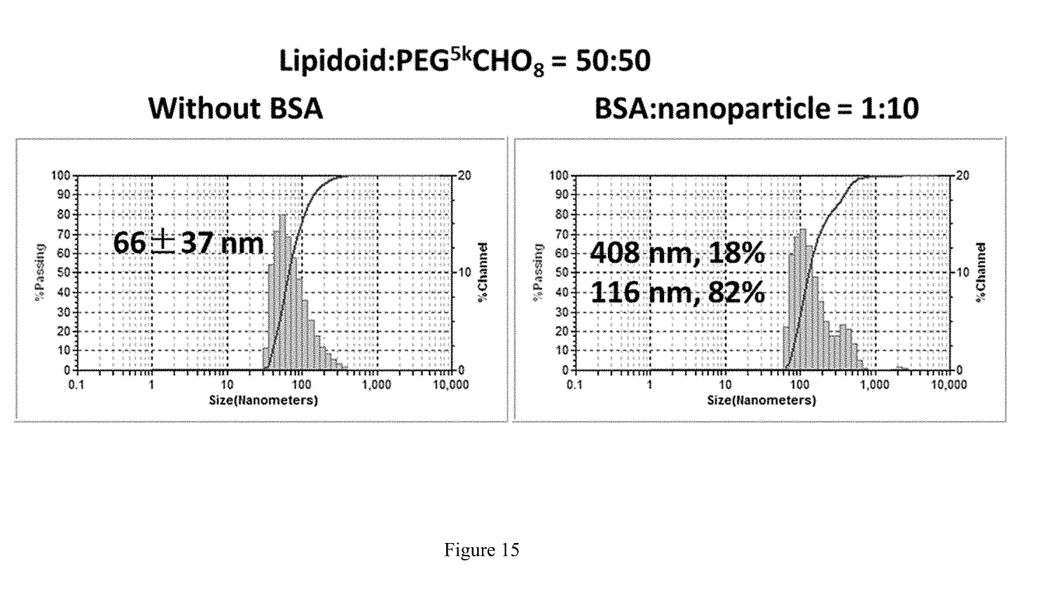

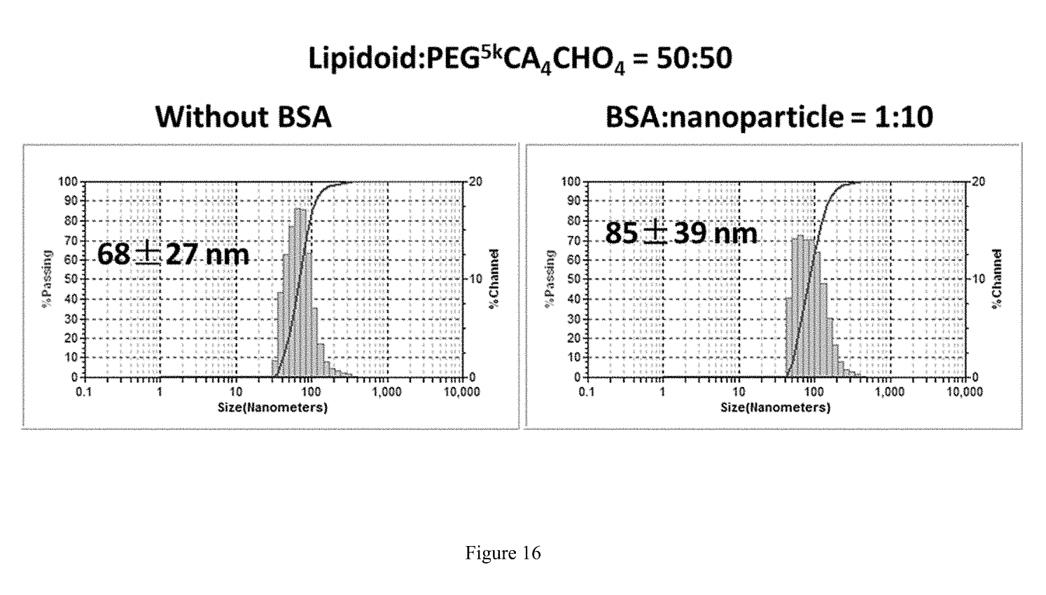

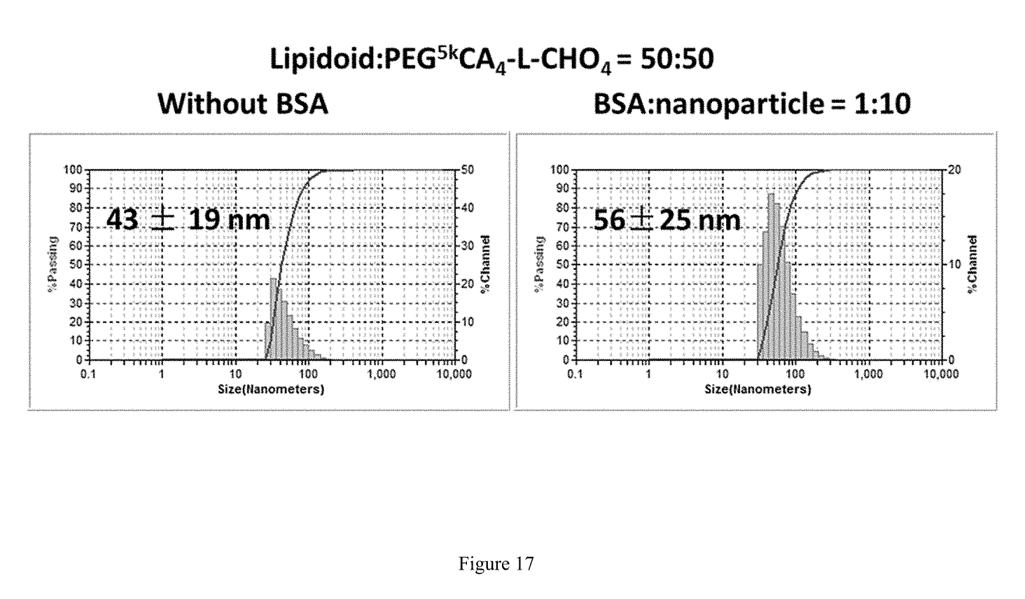

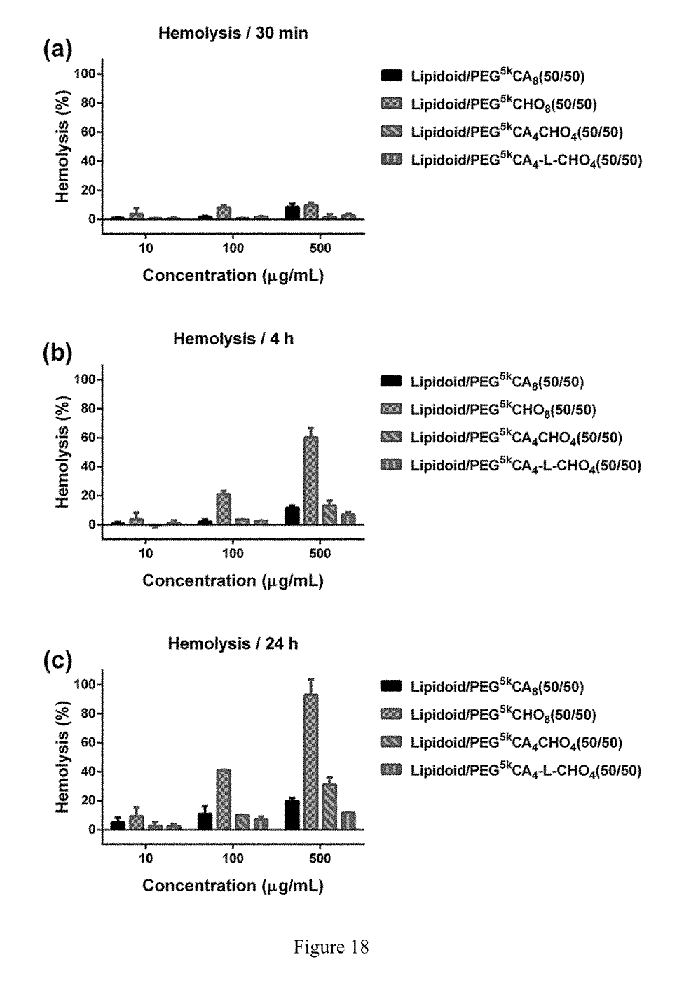

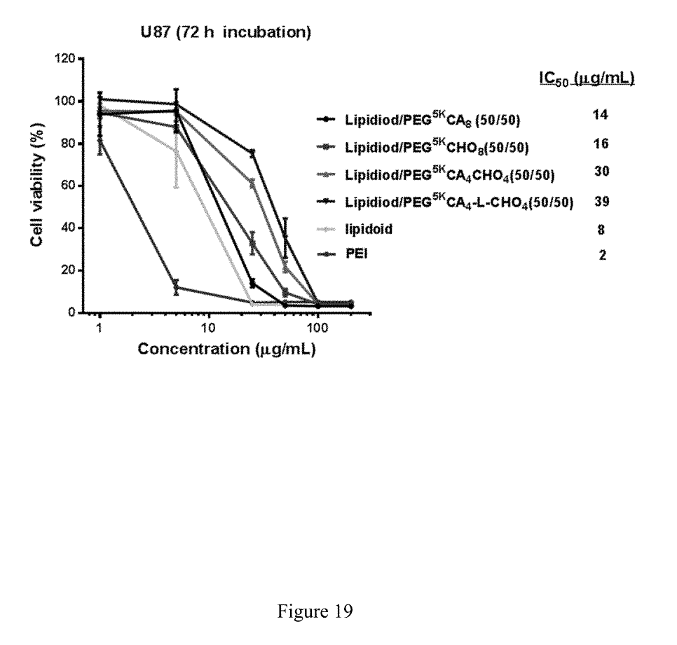

Fabrication of lipidoid-telodendrimer nanoparticles and protein loading. Telodendrimers with diverse compositions and architectures were synthesized through step-wise peptide chemistry, and their chemical structures are displayed in FIGS. 27-29. As a pioneering telodendrimer, PEG^{5k}CA₈ that consists of a linear PEG (M_{w} 5 kDa) and eight amphiphilic cholic acids (CAs) as peripheral groups was first attempted to produce hybrid NPs with the lipidoid. The resulting lipidoid-PEG^{5k}CA₈ NPs with a lipidoid to telodendrimer (L/T) ratio of 50/50 by weight have an average hydrodynamic diameter (Dₕ) of 56±35 nm (Table 1 and FIGS. 14-17) determined by dynamic light scattering (DLS). However, the zeta potential (ζ-potential) of these NPs is over 11 mV, such a highly positive surface charge potential may bring risk for CED. Moreover, large aggregates formed after coupling of a model protein, bovine serum albumin (BSA, M_{w} 66.5 kDa), at a loading ratio of 1/10 (BSA/NP, w/w). The reason is presumably that the amphiphilic CA groups in the telodendrimer are unable to embed effectively into the hydrophobic domains of the lipidoid NPs, leading to a formation of incompact NPs with a large, positive surface charge potential. Hydrophobic cholesterol (CHO) was found to occupy the space between lipid tails naturally in lipid membranes, and it also showed promise to stabilize lipidoid NPs. With this in mind, PEG^{5k}CHO₈ containing eight CHO groups was synthesized, and the properties of lipidoid-PEG^{5k}CHO₈NPs at an L/T ratio of 50/50 by weight before and after loading of BSA were evaluated (Table 1). Though the Dₕ and ζ-potential for lipidoid-PEG^{5k}CHO₈NPs are acceptable, multiple peaks appeared in the hydrodynamic size distribution after loading of BSA at a loading ratio of 1/10 (BSA/NP, w/w). In addition to the unfavorable particle size, the lipidoid-PEG^{5k}CHO₈ NPs exhibited a strong hemolytic activity (FIG. 18), because of the membrane activity of CHO groups and the charge interactions. These facts indicate both lipidoid-PEG^{5k} CA₈ and lipidoid-PEG^{5k}CHO₈NPs are not good candidates for CED of proteins. To optimize the lipidoid NPs, a compromise telodendrimer of PEG^{5k}CA₄CHO₄ was synthesized with four CA groups and four CHO groups on the α- and ε-position of lysine terminus, respectively. NPs generated by the lipidoid and PEG^{5k}CA₄CHO₄ (50/50 of L/T by weight) showed monodispersed hydrodynamic size distribution with a Dₕ of 68±27 nm, a low ζ-potential of 3.4±0.5 mV and a low hemolytic activity (Table 1). After loading of BSA at a mass ratio of 1/10 (BSA/NPs), the particle size increased to 85±39 nm and ζ-potential decreased to −0.3±0.6 mV. These results testify that lipidoid-PEG^{5k}CA₄CHO₄ NPs have better physical properties than lipidoid-PEG^{5k} CA₈ and lipidoid-PEG^{5k}CHO₈NPs mainly because of the joint actions of the amphiphilic CA and hydrophobic CHO groups. Further, a PEG^{5k}CA₄-L-CHO₄ telodendrimer having the segregated CA and CHO domains was designed to enhance the intercalation between telodendrimer via CHO motifs and increase the stability and dispersion of nanocomplex via CA motives. In PEG^{5k}CA₄-L-CHO₄, the CA and CHO groups are segregated by a triethylene glycol diamine derived linker (M_{w} 230). The resulting lipidoid-PEG^{5k}CA₄-L-CHO₄ (50/50 of L/T by weight) NPs before and after loading of proteins exhibited superior properties, including smaller particle size, lower ζ-potential, minimized hemolytic activity (FIG. 18) and reduced cytotoxicity (FIG. 19), when compared to the lipidoid-PEG^{5k}CA₄CHO₄ system (Table 1). In this case, the hydrophilic PEG in the telodendrimer helps to prevent aggregation while the hydrophobic end of CHO groups interacts with the lipidoid tails, and the intermediary amphiphilic CA moiety is able to mediate the difference in polarity between hydrophilic and hydrophobic parts, and endow the NPs with high stability and small size. For example, lipidoid-PEG^{5k}CA₄-L-CHO₄ hybrid system is a promising vehicle for CED of proteins.

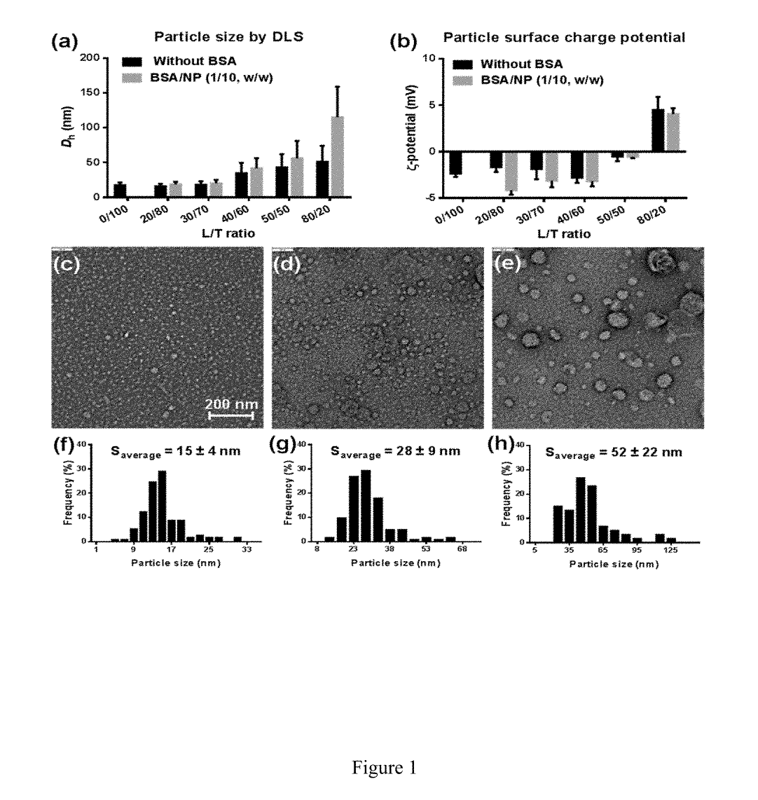

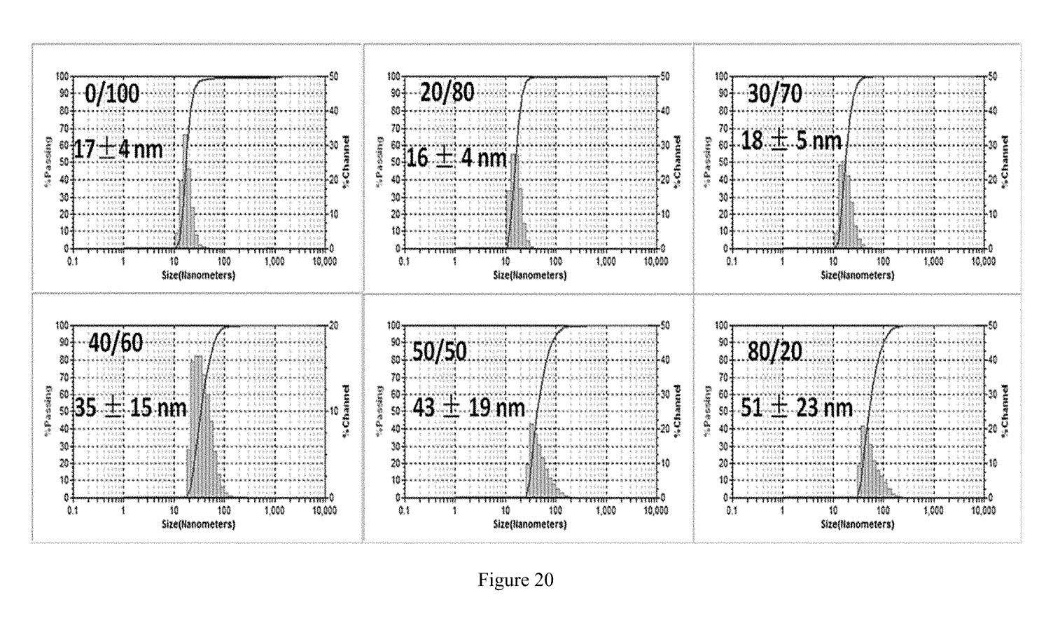

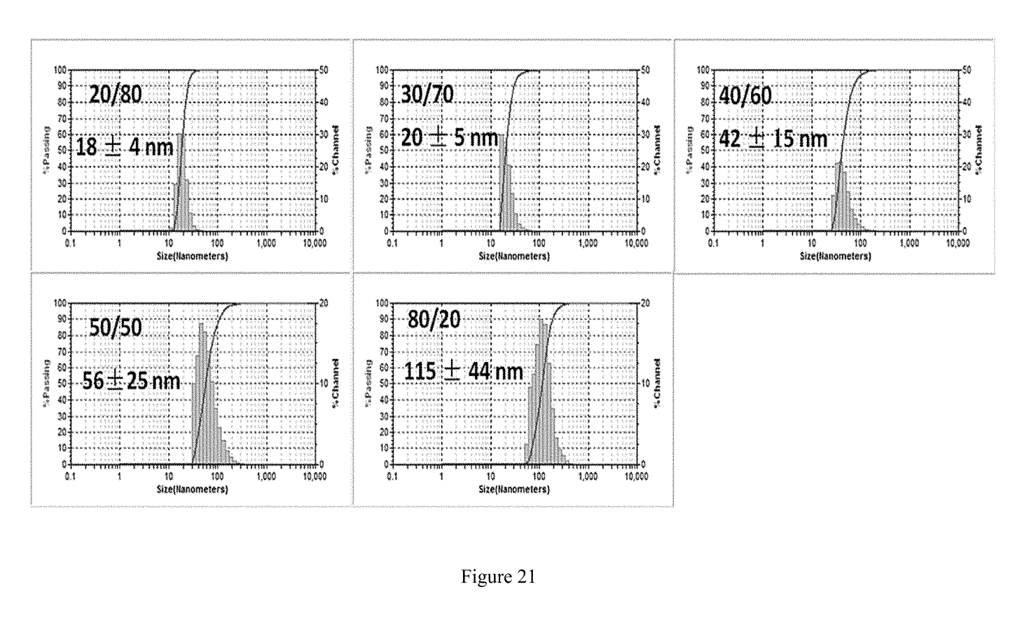

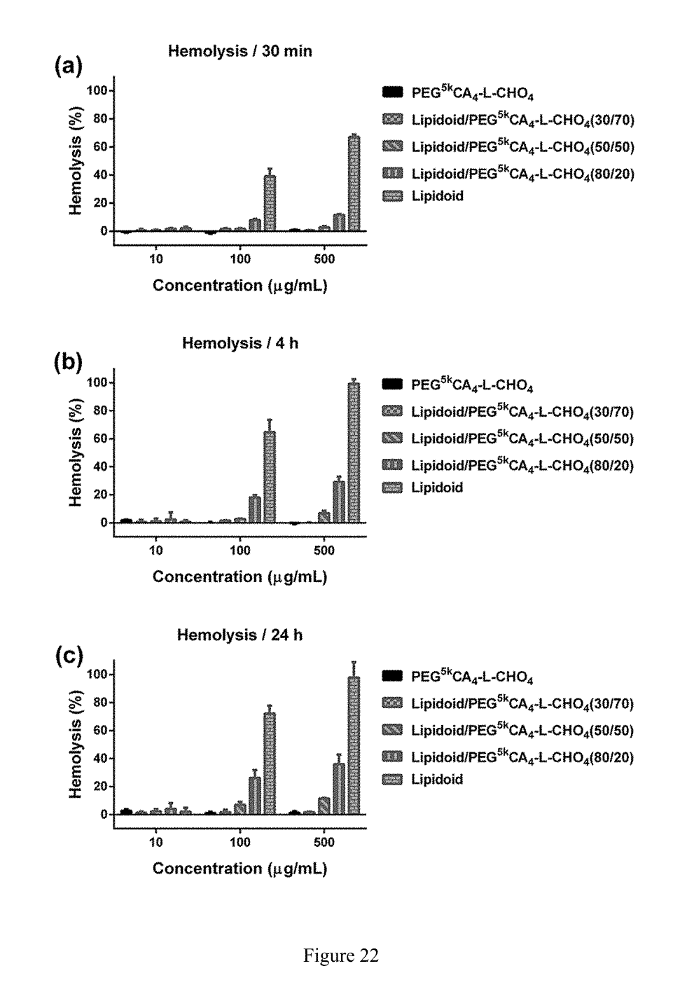

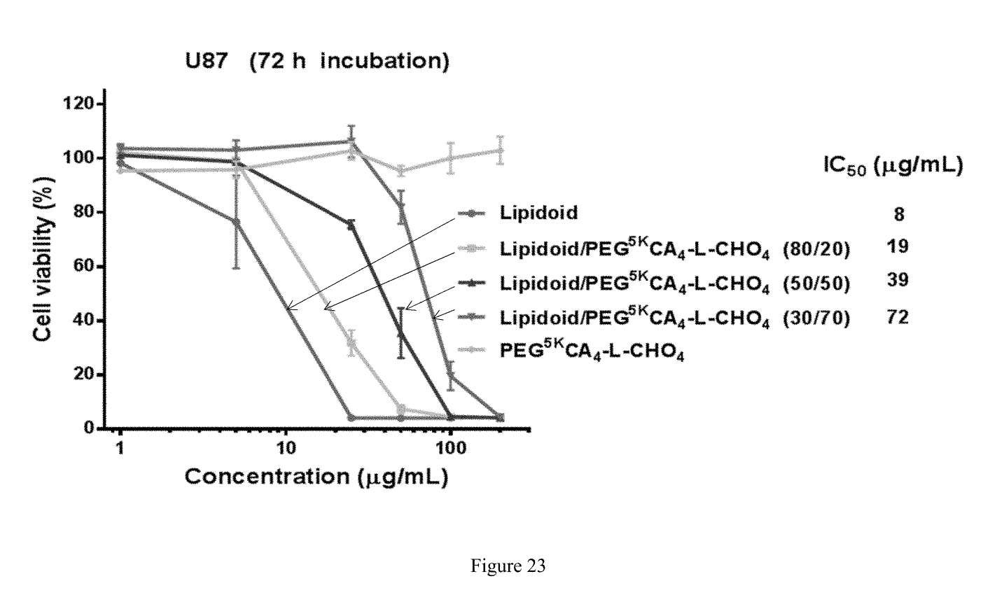

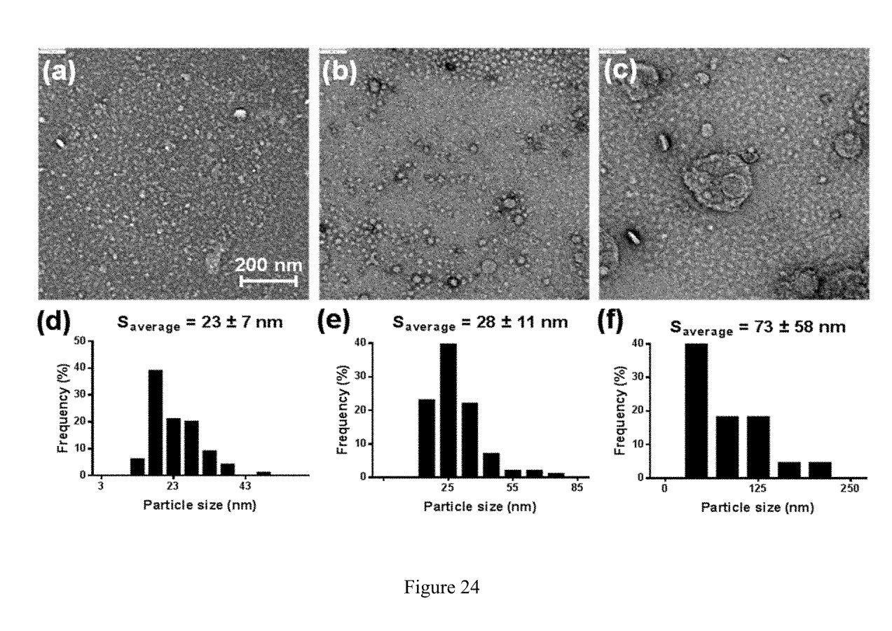

In addition to composition and architecture of the telodendrimers, L/T ratio is another important influence factor to the properties of the hybrid NPs. As shown in FIGS. 1a and 1b, the Dₕ and ζ-potential of the lipidoid-PEG^{5k}CA₄-L-CHO₄ NPs before and after loading of proteins are greatly impacted by L/T ratios. FIGS. 1a and 20 show that the particle sizes increase with increasing lipidoid content due to high hydrophobicity of the lipidoid, which leads to an aggregation state of the NPs. The sizes of the NPs containing less than or equal to 50% wt of lipidoids increase slightly (FIG. 21) after loading of BSA, while the size of the NP with a high lipidoid content (80% wt) increases to over 110 nm after loading of BSA mainly due to less efficient sheltering by telodendrimer to prevent aggregation. The surface charge potential generally increases with increasing lipidoid content owing to the cationic nature of the lipidoid (FIG. 1b). After loading of BSA at a loading ratio of 1/10 (BSA/NPs, w/w), the ζ-potential slightly declined due to the negative net theoretical charge of BSA. The hybrid NPs before and after loading of BSA exhibited negative or neutral ζ-potential when the telodendrimer contents are ≧50%, while the NPs at an L/T ratio of 80/20 showed undesired positive ζ-potential both before and after BSA loading. We also find the doping of telodendrimers to the lipidoid systems can effectively reduce their hemolytic activity and cytotoxicity (FIGS. 22 and 23). Low hemolytic activity and cytotoxicity are observed when the hybrid NP has a lipidoid content of 30% or 50% by weight. The NPs made from lipidoid and PEG^{5k}CA₄-L-CHO₄ at different L/T ratios (30/70, 50/50 and 80/20 by weight) are spherical in shape (FIGS. 1c-e), which was affirmed by transmission electron microscopy (TEM). The average particle sizes that obtained from the TEM images are 15±4, 28±9 and 52±22 nm for 30/70, 50/50 and 80/20 NPs (FIGS. 1f-h), respectively. After loading of BSA at a loading ratio of 10% by weight, the shapes of 30/70 and 50/50 NPs remained spherical, and their average particle sizes changed to 23±7 and 28±11 nm by TEM, respectively. The average particle size of 80/20 NPs increased to 73±58 nm and irregular-shaped large aggregates over 100 nm could be observed after loading of BSA (FIG. 24), which are in reasonable agreement with the DLS results.

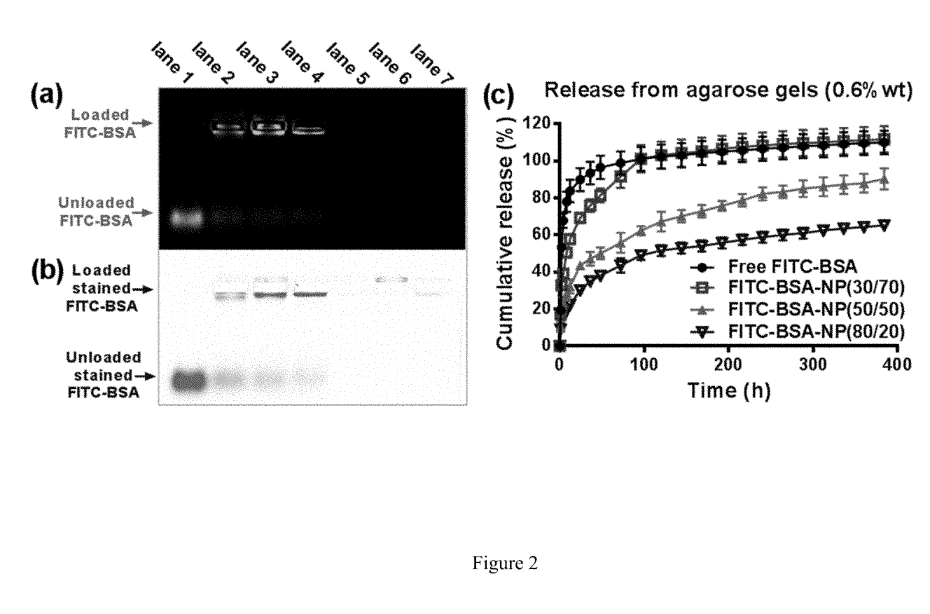

The lipidoid-telodendrimer hybrid NPs can form complexes with proteins based on both electrostatic interactions and hydrophobic-hydrophobic interactions. The loading capacity of the lipidoid-telodendrimer hybrid NPs for a model protein, fluorescein isothiocyanate (FITC) labeled BSA (noted as FITC-BSA), was semi-quantitatively determined by an agarose gel retention assay, where the free FITC-BSA and FITC-BSA-incorporated NPs can be separated based on their differences in size and charge. The fluorescence of FITC-BSA can be visualized in the agarose gel for accurate observation of the protein migration. A constant amount of FITC-BSA without (lane 1 in FIGS. 2a and 2b) or with lipidoid-PEG^{5k}CA₄-L-CHO₄NPs at L/T mass ratios of 30/70 (lane 2), 50/50 (lane 3) and 80/20 (lane 4), and blank NPs (lanes 5-7) were loaded in an agarose gel of a concentration of 1.5% wt, and performed in Tris-acetate-EDTA buffer. After a 90 min (min=minute(s)) of developing, FITC-BSA without NPs migrated a certain distance towards the anode (FIG. 2a), while most FITC-BSA were trapped in the wells when they were conjugated with the NPs due to the large sizes and neutral surface charges of the protein-loaded NPs. Excess FITC-BSA in the protein-NP systems could also migrate a distance equal to that for FITC-BSA without NPs. The non-fluorescent NPs without FITC-BSA could not be observed. The loading capacities of the NPs for FITC-BSA can be calculated from the fluorescence intensities of the bands for unloaded FITC-BSA, and they are 11% (11/100, protein/NP, w/w), 12% and 13% by weight for 30/70, 50/50 and 80/20 NPs, respectively. The gel was then stained by Coomassie blue following by overnight destaining, and the result is presented in FIG. 2b. The loading capacities determined from the intensities of the stained bands for unloaded proteins acquire a good agreement with the results by the fluorescence analysis. The agarose gel at a concentration of 0.6% wt was demonstrated to closely resemble in vivo brain with respect to some critical physical characteristics including the ratio of distribution volume to infusion volume, and the infusion pressure. To mimic the diffusions of free proteins and NP-conjugated proteins in brain tumor, free FITC-BSA and FITC-BSA loaded in lipidoid-PEG^{5k}CA₄-L-CHO₄NPs were dispersed in agarose gels (0.6% wt), which were then immersed in phosphate buffered saline (PBS) and the released FITC-BSA were monitored. Free FITC-BSA released from the gels quickly with approximately 50% of the proteins released within 2 hours (FIG. 2c). In contrast, much slower release profiles were observed for FITC-BSA loaded in the hybrid NPs (˜50% of the proteins released within 8, 48 and 120 hours for the protein-incorporated NPs at L/T mass ratios of 30/70, 50/50 and 80/20, respectively), and the release rate decreased with increasing L/T ratio. This behavior can be attributed to increased particle sizes and strong binding between proteins and NPs. The protein release assays from agarose gels (0.6% wt) suggest that the protein-NP complexes show promise as nanotherapeutics with longer retention time in brain tumor sites after being locally injected when compared to the free proteins.

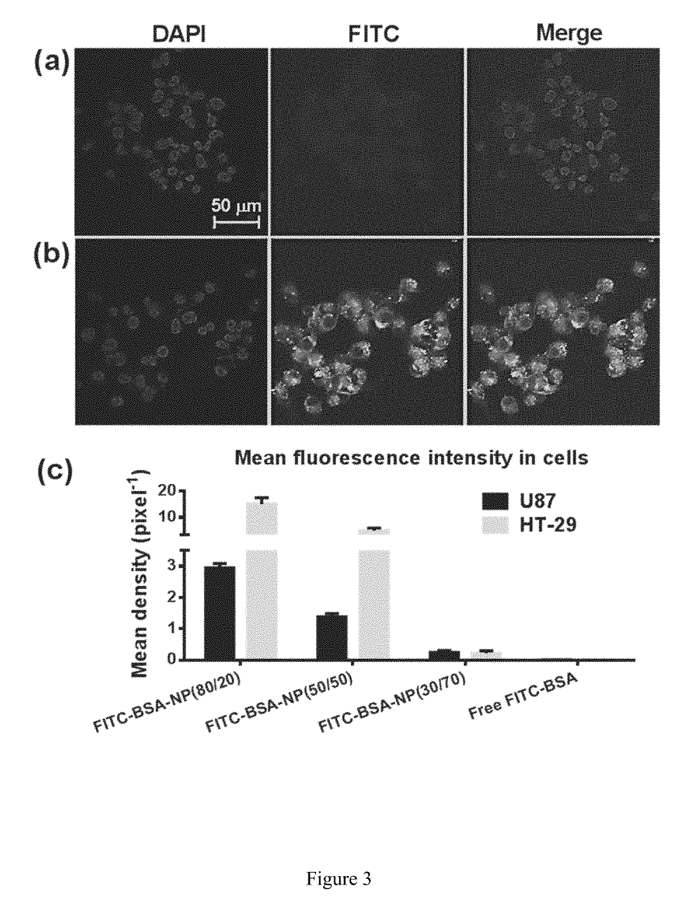

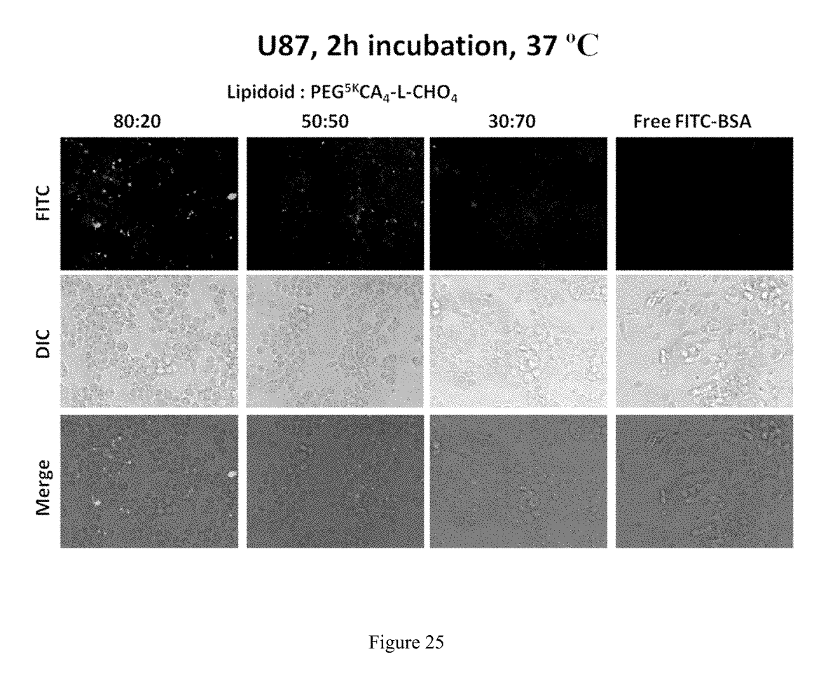

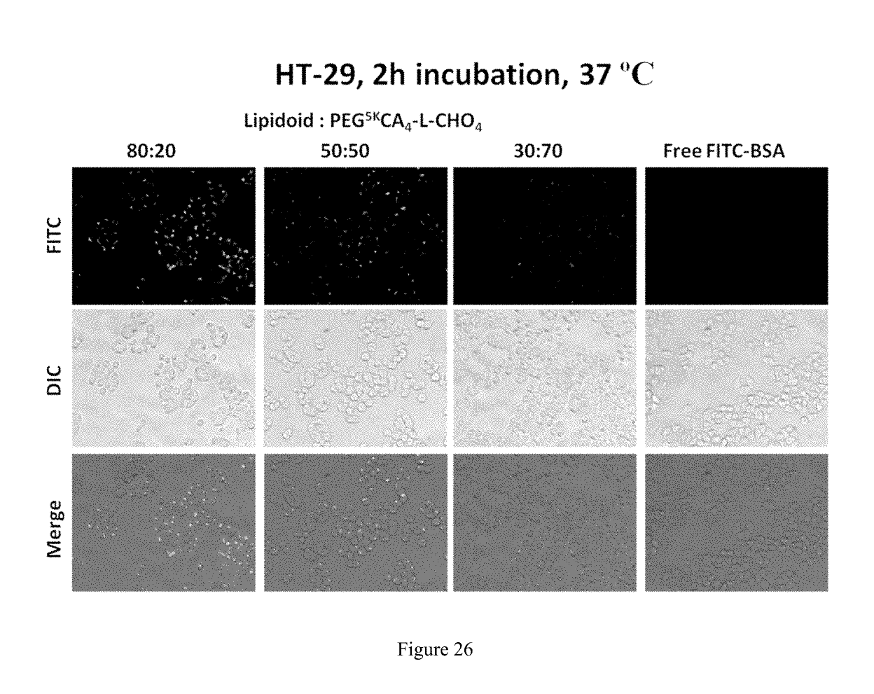

Intracellular delivery of proteins using lipidoid-telodendrimer hybrid nanoparticles. FITC-BSA was used as a fluorescent model protein for probing the intracellular trafficking of proteins without or with lipidoid-telodendrimer hybrid NPs. U87 GBM and HT-29 colon cancer cells were incubated with free FITC-BSA and FITC-BSA-loaded NPs, and observed under fluorescent microscopes. As the confocal laser fluorescence microscopy images revealed in FIG. 3a, the cellular uptake efficiency for free FITC-BSA in U87 cells is very low. In contrast, significant cellular uptake and intracellular accumulation of FITC-BSA-loaded NPs (50/50 of L/T by weight) at a loading ratio of 1/10 (protein/NPs, w/w) can be observed in the cytoplasm of U87 cancer cells (green fluorescence, FIG. 3b), indicating the ability of lipidoid-telodendrimer hybrid NPs for intracellular delivery of proteins. The cellular uptake of either lipidoid NPs or telodendrimer micelles has been proved to follow an energy-dependent endocytosis process, and the protein-loaded lipidoid NPs can efficiently escape from endosome/lysosome after entering cells probably due to the proton sponge effect of cationic lipidoid. FIG. 3c shows a semi-quantitative comparison of cellular uptake efficiency for the large-area microscopy images (FIGS. 25 and 26) of free FITC-BSA and FITC-BSA-loaded lipidoid-PEG5kCA4-L-CHO4 NPs at different L/T ratios (30/70, 50/50 and 80/20, w/w) and at a constant protein loading ratio of 1/10 (protein/NP, w/w) in U87 GBM and HT-29 colon cancer cell lines. The cellular uptake efficiency closes to zero for free FITC-BSA in both U87 and HT-29 cell lines, and the cellular uptake efficiency increases with increasing content of lipidoid in the hybrid NPs. The cellular uptake efficiency of FITC-BSA-loaded NPs at different L/T ratios for HT-29 cells has a similar trend, however, higher than that for U87 cells. These results reveal that the lipidoid-telodendrimer hybrid NPs can serve as valid vehicles for the delivery of proteins into tumor cells.

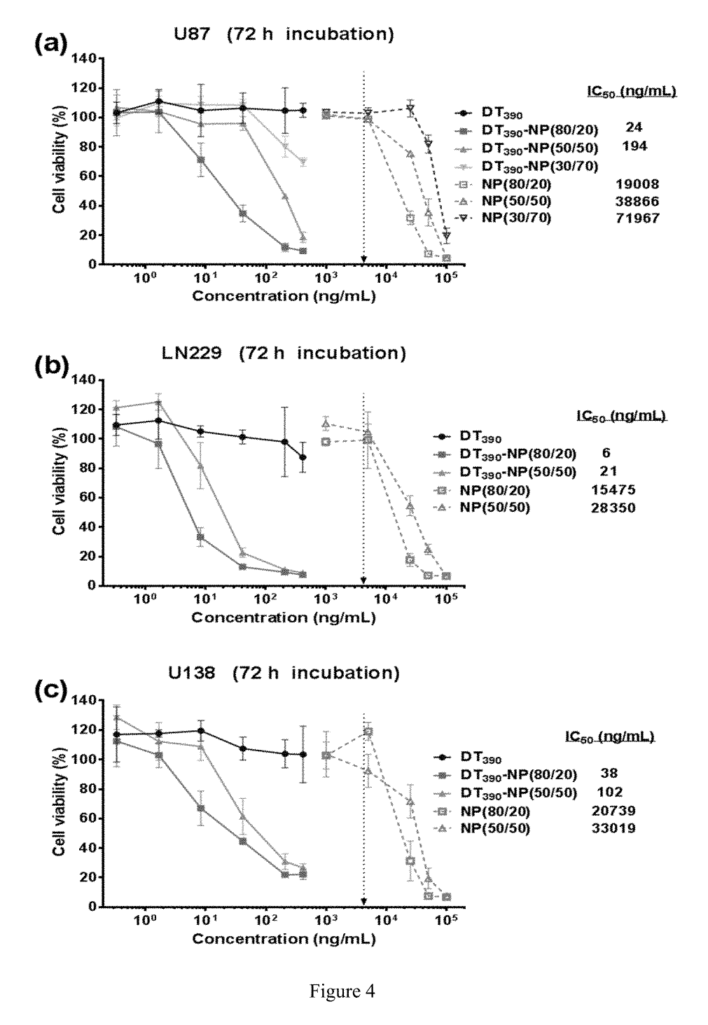

A model therapeutic protein, truncated diphtheria toxin (DT₃₉₀, M_{w} 42.3 kDa), that causes cellular cytotoxicity through the inhibition of protein synthesis after internalization with a potent antitumor effect, was used to investigate the lipidoid-telodendrimer hybrid NP-based intracellular protein delivery. Different GBM cell lines including U87, LN229 and U138 were exposed to free DT₃₉₀ and DT₃₉₀-loaded lipidoid-PEG^{5k}CA₄-L-CHO₄ NPs (1/10 of DT₃₉₀/NP by weight) at different L/T ratios for apoptotic analysis. As shown in FIG. 4, free DT₃₉₀ without NPs show low cytotoxicities against all the cell lines, which are nearly independent on protein concentration. This is because of the ineffective cellular uptake of DT₃₉₀ in the GBM cells. Unlike the free protein, bioactive DT₃₉₀ loaded in lipidoid-PEG^{5k}CA₄-L-CHO₄NPs can be delivered into the cytoplasm of GBM cells resulting in a protein-concentration-dependent killing of these cells, and the killing potency increases with the increasing lipidoid contents in the NPs, which is in reasonable agreement with the cellular uptake results. For U87 cells (FIG. 4a), DT₃₉₀-loaded NPs of a low lipidoid content (30/70 of L/T by weight) exhibit poor potency due to their weak protein delivery efficiency, while DT₃₉₀-loaded hybrid NPs at L/T mass ratios of 50/50 and 80/20 have significantly enhanced protein cytotoxicities with the half-maximal growth inhibitory concentration (IC₅₀) values of 194 and 24 ng/mL. The DT₃₉₀-loaded hybrid NPs at L/T mass ratios of 50/50 and 80/20 also show high potency against other GBM cells, such as LN229 and U138 (FIGS. 4b and 4c). The cytotoxicity assay on various GBM cell lines indicates that no NP-related cytotoxicity is expected in the NP concentrations ranging from 3 to 4,150 ng/mL in these in vitro DT₃₉₀ delivery studies.

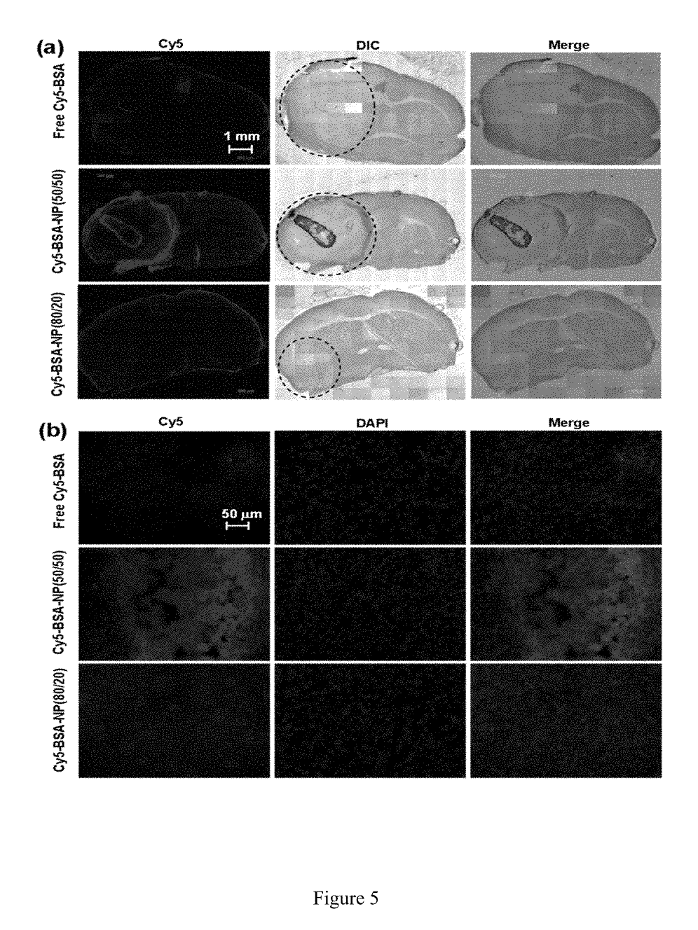

Intracranial distribution of proteins delivered by lipidoid-telodendrimer nanoparticles. The unsatisfied protein delivery efficiency of the lipidoid-PEG^{5k}CA₄-L-CHO₄ NPs at an L/T ratio of 30/70 makes them inadequate for intracellular protein delivery. In contrast, the DT₃₉₀-incorporated NPs at L/T ratios of 50/50 and 80/20 have high potency to kill GBM cells in vitro suggesting the promise to treat GBM tumor in vivo. A near-infrared fluorescence dye-labeled BSA (Cy5-BSA) was used as a model protein to investigate the intracranial distribution of proteins delivered by lipidoid-telodendrimer NPs with the assistant of CED in mouse brains containing orthotopic human U87 GBM tumors. The tumor-bearing mice were sacrificed one day after CED and the brains were acquired and sliced for analysis by confocal microscopy. As shown in FIG. 5a, no obvious fluorescent signals can be detected in the brain sections for the groups treated with free Cy5-BSA and Cy5-BSA-incorporated NPs at an L/T ratio of 80/20, which are presumably because of the leakage of small-sized free proteins from the brain, and the clearance/backflow of the protein-conjugated 80/20 NPs with poor physical properties (including large particle size, positive surface charge, and insufficient PEG coating), respectively. In comparison, Cy5-BSA can be effectively delivered to and accumulated in the brain tumor site within the neutrally charged, sub-100 nm NPs at an L/T ratio of 50/50 (the middle row in FIG. 5a). The uneven distribution of Cy5-BSA in the tumor is mainly due to the highly heterogeneous structure of the GBM tumor. The distributions in the U87 tumors at the cellular level for Cy5-BSA without/with NPs at different L/T ratios (FIG. 5b) also confirm that the NPs with neutral charge and sub-100 nm particle size are good candidates to deliver proteins to intracranial GBM tumors with the assistant of CED.

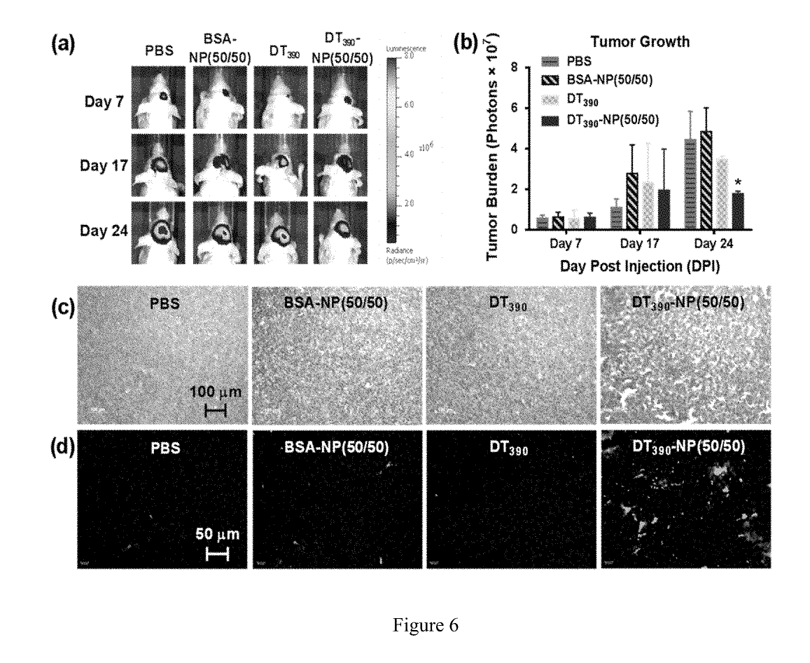

Treatment of brain tumors using protein-loaded lipidoid-telodendrimer hybrid nanoparticles. Based on the in vitro cytotoxicity and in vivo intracranial distribution results, optimized DT₃₉₀-loaded NPs (lipidoid-PEG^{5k}CA₄-L-CHO₄ NPs at an L/T ratio of 50/50 by weight) at a loading mass ratio of protein/NPs of 1/10, that are noted as DT₃₉₀-NPs (50/50), were selected for in vivo brain tumor treatment with the assistant of CED by osmotic pump using a mouse model injected with intracranial U87 tumors, in comparison with free DT₃₉₀. The treatment started at day 9 after the cell implant, and the CED of protein formulations lasted for 7 days. No tumor growth inhibition was found at day 17 post injection (one day after CED of protein formulations), however, comparison of the relative photon counts on tumor sites at day 24 post injection revealed significant differences in tumor volumes between DT₃₉₀-NP treated group and other control groups (FIG. 6a). As shown in FIG. 6b, free DT₃₉₀ and BSA-loaded NP treated groups had similar tumor growth trends to that of PBS control mice. DT₃₉₀-loaded NP-treated mice, however, had reduced photon counts at day 24 post injection and an obvious tumor inhibition when compared to the control groups, indicating the antitumor ability of DT₃₉₀-loaded NPs. The antitumor effect of DT₃₉₀-loaded NPs was further investigated by histological examination. Hematoxylin and eosin (H&E) staining revealed that the remaining tumor mass treated with DT₃₉₀-loaded NPs showed a region with patches of destroyed tumor due to the shrinkage of apoptotic cells (FIG. 6c). In contrast, the dense tumor mass were seen for PBS, BSA-NP, and DT₃₉₀ groups. The terminal deoxynucleotidyl transferased dUTP nick end labeling (TUNEL) assays identified a significant difference in the amount of apoptotic cells (green in FIG. 6d) between the group treated with DT₃₉₀-NPs (50/50) and other control groups. These results suggested the suppression of brain tumor growth by intracranial infusion of therapeutic protein-conjugated lipidoid-telodendrimer hybrid NPs. These results suggested the tumor inhibition effect by the treatment of therapeutic protein-conjugated lipidoid-telodendrimer hybrid NPs.

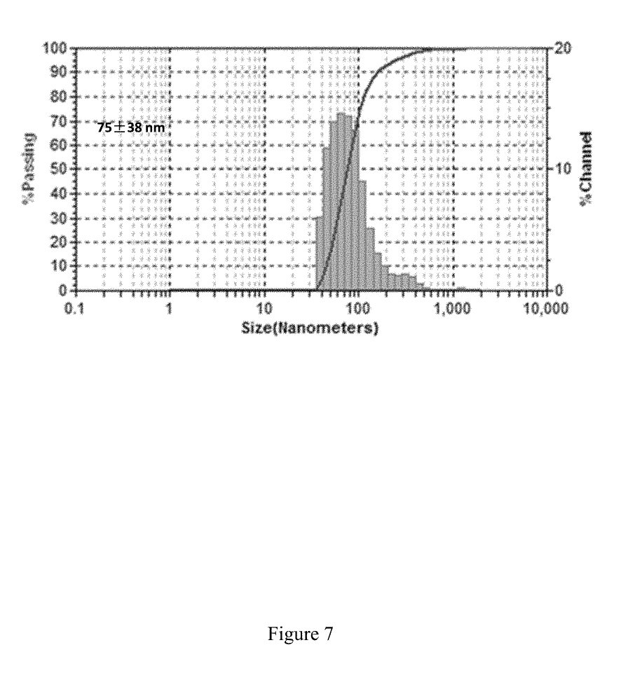

Lipidoid-telodendrimer hybrid nanoparticles for drug loading. The lipidoid-telodendrimer hybrid nanoparticles cannot only conjugate biomacromolecules, but also can load small molecules, such as Mcl-1 inhibitors. The Mcl-1 inhibitor-loaded lipidoid-telodendrimer hybrid nanoparticles (L/T ratio of 80/20, w/w) at a drug loading ratio of 1/10 (drug/nanoparticle, w/w) have a hydrodynamic diameter of ˜75 nm (FIG. 7), which is similar with the blank nanoparticles.

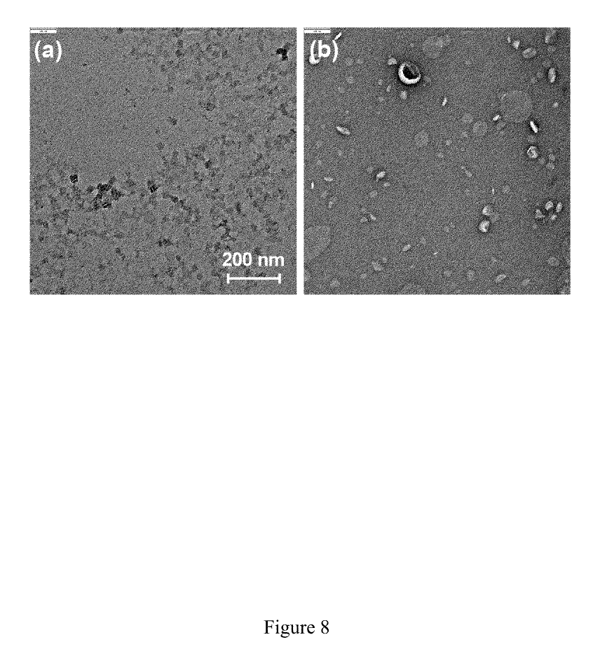

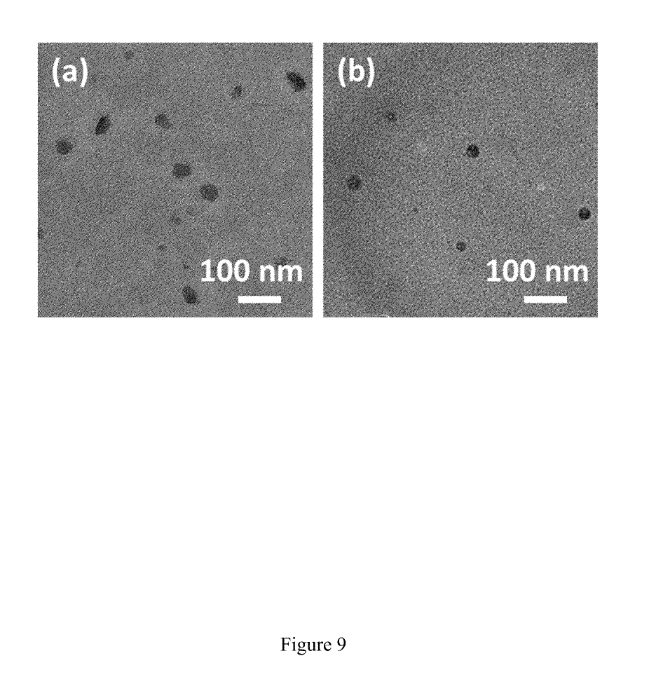

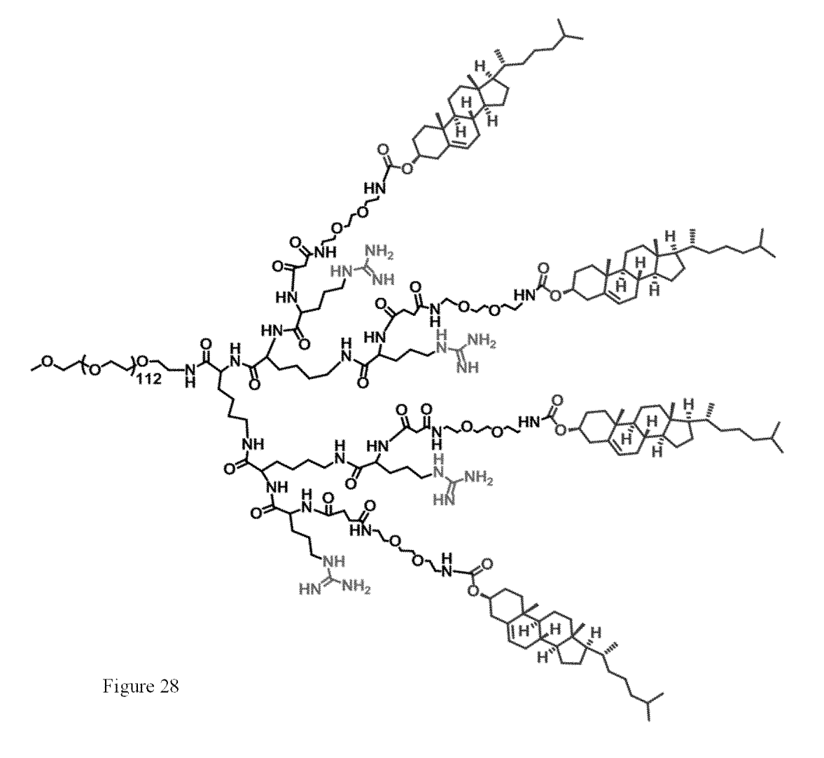

Fabrication of lipid-telodendrimer hybrid nanodiscs for amphotericin B loading. The selection of building blocks for the fabrication of hybrid nanoparticles is not limited to the lipidoid and telodendrimers mentioned above, and it can be extended to other telodendrimers and neutral lipids. For examples, we chose 1,2-dimyristoyl-sn-glycero-3-phosphocholine (DMPC), 1-palmitoyl-2-oleoyl-sn-glycero-3-phosphocholine (POPC), and 1,2-dipalmitoyl-sn-glycero-3-phosphocholine (DPPC) as model lipids, and PEG^{5k}(Arg-L-CHO)₄, PEG^{5k}CA₄-L-CHO₄, and PEG^{5k}CA₄-L-Rh₄ as model telodendrimers to produce lipid-telodendrimer hybrid nanoparticles. The molecular properties of DMPC, POPC and DPPC, including their molecular weights, melting temperatures and chemical structures, are shown in Table 2. The chemical structures of PEG^{5k}(Arg-L-CHO)₄ and PEG^{5k}CA₄-L-Rh₄ telodendrimers are displayed in FIGS. 28 and 29, respectively. As shown in Table 3, the hybrid nanoparticles of DMPC and PEG^{5k}(Arg-L-CHO)₄ at lipid to telodendrimer (L/T) ratios of 1:1 and 5:1 show multiple peaks in the Dₕ distributions while well-defined nanoparticles can be produced when the L/T ratio is 10:1. POPC-PEG^{5k}(Arg-L-CHO)₄ and DPPC-PEG^{5k}(Arg-L-CHO)₄ nanoparticles at various L/T ratios have monodispersed particle size distributions with a Dₕ range from 29 to 75 nm. The TEM images of DPPC-PEG^{5k}(Arg-L-CHO)₄ nanoparticles at L/T ratios of 1:1 (FIG. 8a) and 5:1 (FIG. 8b) indicate that the lipid-telodendrimer hybrid nanoparticles are generally spherical in shape. The ternary systems of lipid(1)-lipid(2)-telodendrimer show similar assembly behaviors with the binary lipid-telodendrimer nanoparticles: the participation of DMPC in the assembly likely induces large aggregation. Specifically speaking, DMPC-POPC-PEG^{5k}(Arg-L-CHO)₄ nanoparticles show multiple peaks in the Dₕ distributions. DMPC-DPPC-PEG^{5k}(Arg-L-CHO)₄ nanoparticles are larger than 400 nm when the lipid(1)/lipid(2)/telodendrimer (L/L/T) ratios are 5:5:1 and 10:10:1 while their sizes are smaller than 50 nm when the L/L/T ratios are 1:1:1 and 2.5:2.5:1. For POPC-DPPC-PEG^{5k}(Arg-L-CHO)₄ nanoparticles, their sizes are smaller than 50 nm. The lipid-telodendrimer system can be used for loading of amphotericin B (AmB), an amphiphilic antifungal drug often used intravenously for serious systemic fungal infections. We expect that the hydrophobic and hydrophilic parts of lipid and telodendrimer will interact with the drug tightly, resulting in stable drug-loaded lipid-telodendrimer nanoparticles. However, after drug loading, all the lipid-PEG^{5k}(Arg-L-CHO)₄ systems show precipitations. Given the same hydrophobic groups in the telodendrimers, we found the nonionic system of PEG^{5k}CA₄-L-CHO₄ performed better than the ionic PEG^{5k}(Arg-L-CHO)₄ in the lipid-telodendrimer system. DMPC and DPPC, as well as the mixture of DMPC and DPPC, can form well-defined hybrid nanoparticles with PEG^{5k}CA₄-L-CHO₄ (Table 3), especially at high L/T ratios. After drug loading, DMPC- and DMPC/DPPC-containing systems still show well-dispersed particle size distributions while DPPC-containing system show precipitations. However, after one month of storage in room temperature, precipitations were found in all of the solution of these PEG^{5k}CA₄-L-CHO₄-containing nanoformulations. This shortage can be overcome by using PEG^{5k}CA₄-L-Rho as building blocks for lipid-telodendrimer hybrid nanoparticle construction. The DMPC-PEG^{5k}CA₄-L-Rh₄ hybrid nanoparticles at an L/T ratio of 1/5 (w/w) show well-defined particle sizes before and after AmB loading (FIG. 9), and they are colloidally stable for one month storage (Table 3), indicating their promising application for AmB delivery.

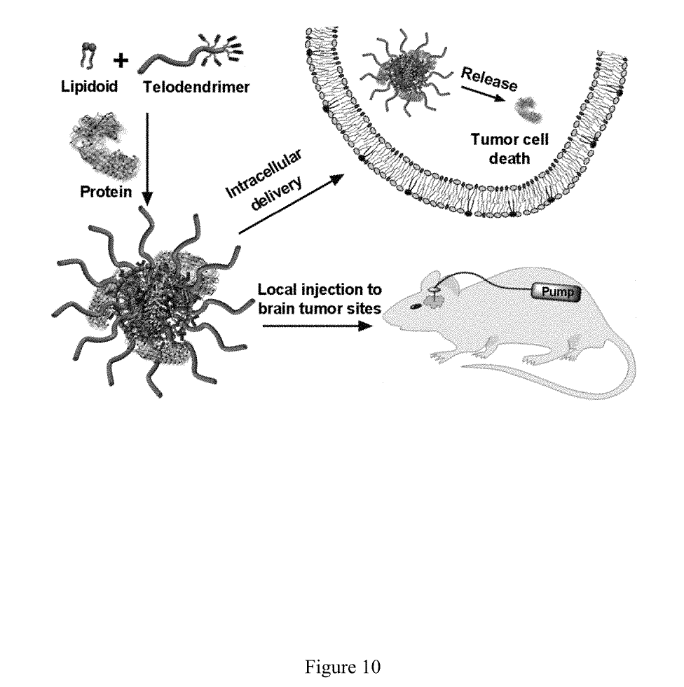

We have established a facile strategy to create precisely engineer-able lipid-like nanoparticles employing telodendrimers as building blocks for intracellular delivery of therapeutic proteins or small molecule drugs used in brain cancer treatment or other applications (FIG. 10). The physical properties of the nanoparticles such as particle size and surface charge potential, as well as their biochemical properties including hemolytic activity, cytotoxicity and intracellular protein delivery efficiency can be simply controlled by adjusting the architecture/composition of the telodendrimers and the L/T ratio. The optimized nanoparticles with a particle size of sub-100 nm and neutral ζ-potential can potently deliver therapeutic proteins to intracranial cancer cell interior by the assistant of CED while maintaining protein bioactivity, resulting in brain tumor inhibition. We expect the current matrices of lipidoid-telodendrimer hybrid nanoparticles are not only useful for brain tumor therapy, but also promising to serve as platforms used for the treatment of other diseases, owing to the desirable properties of the nanoparticles. Our study demonstrates the significant impact of the precise control on polymers at molecular level to the properties of lipidoid (or lipid)-polymer hybrid nanoparticles, which may serve as a guide for the bottom-up rational design of engineer-able nanoparticles.

TABLE 1
Properties of lipidoid-telodendrimer hybrid NPs
(50/50 of L/T by weight) and protein-loaded NPs.

| | | | | | Dₕ (nm) | ζ-potential |
|---|---|---|---|---|---|---|
| telodendrimer in | Dₕ | ζ-potential | hemolysis | IC₅₀ | with | (mV) with |
| hybrid NP | (nm)^{a} | (mV)^{a} | (%)^{b} | (μg/mL)^{c} | protein^{d} | protein^{d} |
| PEG^{5k}CA₈ | 56 ± 35 | 11.4 ± 1.6 | 2.1 ± 1.7 | 14 | multiple | 2.6 ± 1.1 |
| PEG^{5k}CHO₈ | 66 ± 37 | 0.6 ± 0.5 | 21.0 ± 2.1 | 16 | multiple | −1.1 ± 0.7 |
| PEG^{5k}CA₄CHO₄ | 68 ± 27 | 3.4 ± 0.5 | 3.6 ± 0.3 | 30 | 85 ± 39 | −0.3 ± 0.6 |
| PEG^{5k}CA₄-L-CHO₄ | 43 ± 19 | −0.5 ± 0.5 | 2.6 ± 0.4 | 39 | 56 ± 25 | −0.6 ± 0.1 |

^{a}Obtained at a concentration of 0.2 mg/mL in PBS.
^{b}Acquired at 4 h after the diluted red blood cell suspension was mixed with the NPs (0.1 mg/mL).
^{c}Obtained after a 72 h continuous incubation on U87 cells.
^{d}Acquired after incorporation of BSA (1/10 of protein/NP, w/w) in PBS with a NP concentration of 0.2 mg/mL.

Table 2
Molecular properties of the lipids.

| | Mw | Tₘ | |
|---|---|---|---|
| Lipid | (g/mol)^{a} | (° C.)^{b} | Chemical structure |
| DMPC | 677.93 | 24 | 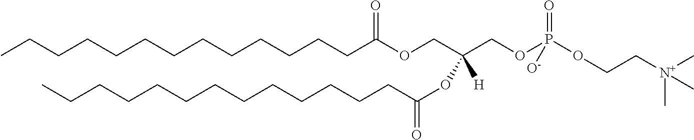 [SMILES: CCCCCCCCCCCCCC(=O)OC[C@H](COP(=O)([O-])OCC[N+](C)(C)C)OC(=O)CCCCCCCCCCCCC] |
| POPC | 760.08 | −2 | 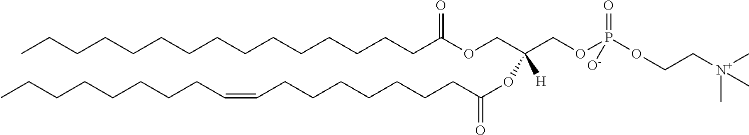 [SMILES: CCCCCCCC/C=C\CCCCCCCC(=O)O[C@H](COC(=O)CCCCCCCCCCCCCCC)COP(=O)([O-])OCC[N+](C)(C)C] |
| DPPC | 734.04 | 41 | 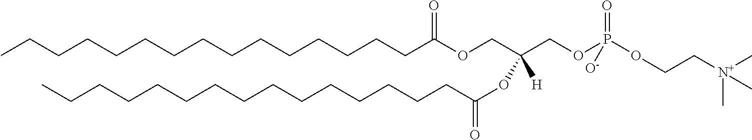 [SMILES: CCCCCCCCCCCCCCCC(=O)OC[C@H](COP(=O)([O-])OCC[N+](C)(C)C)OC(=O)CCCCCCCCCCCCCCC] |

^{a}Molecular weight (Mw) of the lipid.
^{b}Melting temperature (Tₘ) of the lipid.

TABLE 3
Hydrodynamic diameters of the hybrid nanoparticles of lipids and
telodendrimers before and after loading of AmB at a loading ratio of 1/10 (drug/nanoparticle).

| | | | | | Dₕ with AmB |
|---|---|---|---|---|---|
| | | | | | after storage |
| | | Mass | | Dₕ with AmB | for 1 month |
| Lipid(s) | Telodendrimer | ratio | Dₕ (nm) | (nm) | (nm) |
| DMPC | PEG^{5k}(Arg-L-CHO)⁴ | 1:1 | multiple peaks | — | — |
| | | 5:1 | multiple peaks | — | — |
| | | 10:1 | 67 ± 33 | precipitation | — |
| POPC | PEG^{5k}(Arg-L-CHO)₄ | 1:1 | 51 ± 19 | precipitation | — |
| | | 5:1 | 39 ± 14 | precipitation | — |
| | | 10:1 | 33 ± 14 | precipitation | — |
| DPPC | PEG^{5k}(Arg-L-CHO)₄ | 1:1 | 29 ± 10 | precipitation | — |
| | | 5:1 | 75 ± 32 | precipitation | — |
| | | 10:1 | 37 ± 17 | precipitation | — |
| DMPC + POPC | PEG^{5k}(Arg-L-CHO)₄ | 0.6:0.6:1 | multiple peaks | — | — |
| | | 1:1:1 | multiple peaks | — | — |
| DMPC + DPPC | PEG^{5k}(Arg-L-CHO)₄ | 1:1:1 | 28 ± 12 | precipitation | — |
| | | 5:5:1 | 491 ± 190 | precipitation | — |
| | | 10:10:1 | 452 ± 186 | precipitation | — |
| | | 2.5:2.5:1 | 49 ± 20 | precipitation | — |
| POPC + DPPC | PEG^{5k}(Arg-L-CHO)₄ | 0.6:0.6:1 | 31 ± 13 | precipitation | — |
| | | 1:1:1 | 43 ± 25 | precipitation | — |
| DMPC + DPPC | PEG^{5k}CA₄-L-CHO₄ | 0.6:0.6:1 | 30 ± 10 | 53 ± 22 | precipitation |
| | | 2.5:2.5:1 | 35 ± 12 | 45 ± 18 | precipitation |
| DPPC | PEG^{5k}CA₄-L-CHO₄ | 1:1 | multiple peaks | — | — |
| | | 1:5 | 48 ± 22 | precipitation | — |
| DMPC | PEG^{5k}CA₄-L-CHO₄ | 1:1 | multiple peaks | — | — |
| | | 3:1 | 54 ± 28 | 45 ± 19 | precipitation |
| | | 5:1 | 44 ± 18 | 54 ± 22 | precipitation |
| DMPC | PEG^{5k}CA₄-L-Rh₄ | 1:1 | multiple peaks | — | — |
| DMPC | PEG^{5k}CA₄-L-Rh₄ | 3:1 | multiple peaks | — | — |
| DMPC | PEG^{5k}CA₄-L-Rh₄ | 5:1 | 87 ± 35 | 58 ± 31 | 83 ± 37 |

EXPERIMENTAL SECTION. Materials. Monomethylterminated poly(ethylene glycol) monoamine hydrochloride (MeO-PEG-NH₂.HCl, M_{w}: 5 kDa) was purchased from Jenkem Technology. (Fmoc)Lys(Fmoc)-OH was obtained from AnaSpec Inc. Cholesteryl chloroformate was purchased from Alfa Aesar. 1,2-Epoxyhexadecane was obtained from TCI America. N,N′-Dimethyl-1,3-propanediamine and amphotericin B were purchased from Acros Organics. CellTiter 96® A_{Queous} MTS reagent powder was purchased from Promega. Cholic acid (CA), diisopropylcarbodiimide (DIC), N-hydroxybenzotriazole (HOBt), N-hydroxysuccinimide (HOSu), N,N-diisopropylethylamine (DIEA), fluorescein isothiocyanate isomer I (FITC), bovine serum albumin (BSA, M_{w} 66.5 kDa, isoelectric point 5.4), polyethylenimine (PEI, branched, M_{w} 25 kDa), rhein and other chemical reagents were purchased from Sigma-Aldrich. The lipids of 1,2-dimyristoyl-sn-glycero-3-phosphocholine (DMPC), 1-palmitoyl-2-oleoyl-sn-glycero-3-phosphocholine (POPC), and 1,2-dipalmitoyl-sn-glycero-3-phosphocholine (DPPC) were purchased from Avanti Polar Lipids, Inc. Dialysis membrane with 3,500 M_{w} cut off was purchased from Spectrum Laboratories, Inc. Truncated diphtheria toxin (DT₃₉₀, M_{w} 42.3 kDa, isoelectric point 5.1) was provided by Dr. Walter A. Hall of Department of Neurosurgery at State University of New York Upstate Medical University. The sequence of DT₃₉₀ is listed as follow:

(SEQ ID NO: 1)
GADDVVDSSKSFVMENFSSYHGTKPGYVDSIQKGIQKPKSGTQGNYDDD
WKGFYSTDNKYDAAGYSVDNENPLSGKAGGVVKVTYPGLTNVLALKVDN
AETIKKELGLSLTEPLMEQVGTEEFIKRFGDGASRVVLSLPFAEGSSSV
EYINNWEQAKALSVELEINFETRGKRGQDAMYEYMAQACAGNRVRRSVG
SSLSCINLDWDVIRDKTKTKIESLKEHGPIKNKMSESPNKTVSEEKAKQ
YLEEFHQTALEHPELSELKTVTGTNPVFAGANYAAWAVNVAQVIDSETA
DNLEKTTAALSILPGIGSVMGIADGAVHHNTEEIVAQSIALSSLMVAQA
IPLVGELVDIGFAAYNFVESIINLFQVVHNSYNRPAYSPGHKTQPF

Telodendrimer Synthesis. The telodendrimers were synthesized using a solution-phase condensation reaction starting from MeO-PEG-NH₂.HCl (5 kDa) via stepwise peptide chemistry using DIC/HOBt or HOSu/DIEA as coupling agents. The synthesis and characterization of these telodendrimers was carried out using known methods.

Lipidoid Synthesis. The lipidoid (its chemical structure as shown in the inset of FIG. 11) was synthesized according to literature methods. Briefly, in a 10 mL glass vial, 1,2-epoxyhexadecane and NN-dimethyl-1,3-propanediamine were mixed at molar ratios of 2.4:1, following by a two-day reaction at 80° C. without solvent. After cooling to room temperature, the reaction mixture was purified through flash chromatography on silica gel, and characterized by ¹H NMR and MALDI-TOF mass spectrometry.

Formulation of Lipidoid-Telodendrimer Nanoparticles. Nanoparticles were formed by mixing lipidoids and/or telodendrimers at required mass ratios in a solution of 90% ethanol and 10% 10 mM sodium citrate (by volume). Particle solutions were diluted with 10 times volume of phosphate buffered saline (PBS, 1×), and dialyzed against PBS (1×) for 4 h. The particle solution was then mixed with proteins at a ratio of 1/10 (protein/nanoparticle, w/w) to form nanoparticle-protein complex. The complex solution was then stored in sealed vessels at 4° C. for 1 day before use. For Mcl-1 inhibitor-loaded nanoparticle preparation, the Mcl-1 inhibitor, lipidoids and telodendrimers were in a solution of 90% ethanol and 10% 10 mM sodium citrate (by volume). Particle solutions were diluted with 10 times volume of PBS, yielding Mcl-1 inhibitor-loaded lipidoid-telodendrimer nanoparticles.

Formulation of Lipid-Telodendrimer Nanoparticles for Amphotericin B Loading. Thin film and hydration method was used to prepare lipid-telodendrimer nanoparticles without/with amphotericin B. Briefly, the lipids, telodendrimers, and drugs were dissolved in 5 mL of methanol/CHCl₃ (1/1, v/v) solution with 10 μL of triethylamine, and the organic solvents were removed under vacuum to form a thin film of uniform mixture of lipids, telodendrimers and drugs. The thin film was then hydrated in PBS to form the lipid-telodendrimer nanoparticles without/with amphotericin B loaded.

Fluorescently Labeled Proteins. FITC-labeled BSA (named as FITC-BSA) was synthesized according to literature methods. Briefly, FITC-BSA was prepared by mixing 3 mg of FITC dissolved in 0.3 mL of DMSO with 10 mL of BSA aqueous solution (10 mg/mL) in the presence of 0.1 M of NaHCO3 under stirring. The molar ratio of FITC to BSA is approximately 5:1. After 24 h, the reaction mixture was dialyzed against deionized water in the dark for one week to remove the unreacted FITC molecules.

Agarose Gel Retention Assay. FITC-BSA (1 mg/mL), FITC-BSA-incorporated nanoparticles (1 mg/mL for FITC-BSA, and 5 mg/mL for nanoparticles), and blank nanoparticles (5 mg/mL) in loading buffer (30% glycerol aqueous solution) were loaded into an agarose gel of a concentration of 1.5% wt in Tris-acetate-EDTA (TAE) buffer (1×). The gel tray was run for 90 min at a constant current of 20 mA. The gel was imaged by a Bio-Rad Universal Hood II Imager (Bio-Rad Laboratories, Inc.). The loading capacities of nanoparticles were first calculated from the Adj. Vol. (Int.) of the fluorescence bands for free FITC-BSA using the Image Lab 3.0 software. The gel was then stained with 1% Coomassie blue (30 min) followed by overnight destaining. The loading capacities of nanoparticles were recalculated from the Adj. Vol. (Int.) of the stained bands for free FITC-BSA using the Image Lab 3.0 software. The loading capacities of nanoparticles for FITC-BSA calculated by the fluorescence bands and the stained bands are almost identical.

Protein Release from Agarose Gels. Free FITC-BSA and FITC-BSA-incorporated lipidoid-telodendrimer hybrid nanoparticles (1/10, FITC-BSA/nanoparticle, w/w) were loaded in agarose gels of a concentration of 0.6% wt. The sample-loaded agarose gels were immersed in PBS (1×) with shaking for protein release assays. The concentration of released FITC-BSA at various time points was measured by fluorescence spectroscopy employing a pre-established calibration equation. The release medium was replaced with fresh medium. The accumulated protein release was reported as the means for each triplicate sample.

Hemolytic Assays. Hemolysis studies were conducted according to previous methods. One milliliter of fresh blood from healthy human volunteers was collected into 5 mL of PBS (1×) solution in the presence of 20 mM EDTA. Red blood cells (RBCs) were then separated by centrifugation at 1,000 rpm for 10 min. The RBCs were washed three times with 10 mL of PBS (1×) and resuspended in 20 mL of PBS (1×). Diluted RBC suspension (200 μL) was mixed with nanoparticle PBS (1×) solutions or lipidoid DMSO solutions at serial concentrations (10, 100, and 500 μm/mL) by gentle vortex and incubated at 37° C. After 0.5 h, 4 h, and overnight, the mixtures were centrifuged at 1,000 rpm for 5 min. The supernatant free of hemoglobin was determined by measuring the absorbance at 540 nm using a UV-vis spectrometer. Incubations of RBCs with Triton-100 (2%) and PBS or PBS-DMSO mixture (40/1, v/v) were used as the positive and negative controls, respectively. The percent hemolysis of RBCs was calculated using the following formula:

$$RBC hemolysis = \frac{( {OD}_{sample} - {OD}_{negative control} )}{( {OD}_{positive control} - {OD}_{negative control} )} \times 100 \% \qquad ( 1 )$$

Cell Culture and MTS Assays. The human glioblastoma multiforme (GBM) cell lines U87 and LN229, as well as the colon cancer cell line HT-29 cell line were purchased from American Type Culture Collection (ATCC, Manassas, Va., U.S.A.). U138 cell line established from human patients diagnosed with GBM. All cells were cultured in McCoy's 5 A medium supplemented with 10% fetal bovine serum (FBS), 100 U/mL penicillin G, and 100 μm/mL streptomycin at 37° C. using a humidified 5% CO₂ incubator. Various formulations of proteins with different dilutions were added to the plate and then incubated in a humidified 37° C., 5% CO₂ incubator. After a 72 h continuous incubation, a mixture solution composed of CellTiter 96 AQueous MTS, and an electron coupling reagent, PMS, was added to each well according to the manufacturer's instructions. The cell viability was determined by measuring absorbance at 490 nm using a microplate reader (BioTek Synergy 2). Untreated cells served as the control. Results were shown as the average cell viability [100%×(ODₜᵣₑₐₜ−OD_{blank})/(OD_{control}−OD_{blank})] of triplicate wells. The cells were also treated with blank nanoparticles in PBS (1×) and blank lipidoids in a mixture of cremophor/ethanol (v:v=1:1) with different dilutions and incubated for a total of 72 h to evaluate nanoparticle-related toxicity.

Cellular Uptake of Protein-Incorporated Nanoparticles. The cellular uptake and intracellular trafficking of the protein-incorporated nanoparticles were determined by fluorescence microscopy. FITC-BSA was used as a model protein. HT-29 and U87 cells were seeded in chamber slide with a density of 5×10⁴ cells per well in 350 μL of McCoy's 5 A and cultured for 24 h. The original medium was replaced with free FITC-BSA and FITC-BSA-loaded nanoparticles at final FITC a concentration of 1 or 3 μg/mL at 37° C. After a 2 h incubation, the cells were washed three times with cold PBS (1×) and fixed with 4% formaldehyde for 10 min at room temperature, and the cell nuclei were stained with DAPI. The slides were mounted with cover slips and cells were imaged with a Leica fluorescence microscope or a Nikon FV1000 laser scanning confocal scanning microscope. The mean fluorescence density in cells (unit: pixel⁻¹) was calculated using the following equation:

$$Mean fluorescence density in cells = \frac{{A}_{image} \times {D}_{image}}{{A}_{cell}} \qquad ( 2 )$$

where A_{image} is the pixel area of image (unit: pixel²), D_{image} is the mean fluorescence density of image (unit: pixel⁻¹), and A_{cell} is the pixel area of cells (unit: pixel²). These parameters were obtained by analysis of the microscopy images using the ImageJ software.

Intracranial Orthotopic GBM Tumor Model. Female athymic nude mice (NCRNU-Sp/Sp), 6-7 weeks age, were purchased from Taconic Biosciences (Germantown, N.Y.). All animals were kept under pathogen-free conditions according to Association for Assessment and Accreditation of Laboratory Animal Care (AAALAC) guidelines and were allowed to acclimatize for at least 4 days prior to any experiments. All animal experiments were performed in compliance with institutional guidelines and according to protocol approved by the Committee for the Humane Use of Animals of State University of New York Upstate Medical University. Athymic nude mice were anesthetized with ketamine/xylazine (80 mg/kg: 5 mg/kg) through an intraperitoneal injection. The mice were then placed in a stereotactic head frame. Using aseptic technique, a midline skin incision was made a 0.5 mm anterior to bregma and 2.5 mm lateral of midline, a burr hole will be placed. A 25G needle delivered U87 (GBM) tumor cells 3.2 mm deep via the stereotactic head frame. Approximately 5×10⁴ cells (in 1 μL of PBS) will be injected. This injection will occur over the course of 5 minutes. Bone wax will be used to seal the burr hole and glue used to close the skin. Bioluminescence imaging was used to monitor the tumor growth and to observe changes in tumor size after injection of therapeutic NP formulations.

CED Administration of Protein Formulations. Tumor-bearing mice received CED for intracranial distribution and intracranial tumor growth inhibition studies. Free protein and protein-incorporated NPs (2 μg of proteins in 100 μL of PBS for each mouse, 1/10 of protein/NP by weight) were infused at a rate of 0.6 μL/h over 7 days by a sterile osmotic pump for CED. Controls were infused with PBS. In vivo bioluminescence imaging studies were carried out using IVIS 50 (PerkinElmer). For imaging, mice with intracranial U87 tumor were simultaneously anesthetized with isoflurane, and D-luciferin potassium salt was intraperitoneally administered at a dose of 3.75 mg/mouse. For bioluminescence image analysis, the associated bioluminescence intensities were determined by Living Image software (Caliper Life Sciences) using operator-defined regions of interest (ROI) measurements.

Immunohistochemistry and in Vivo Apoptosis Assay. Mice were sacrificed after treatment with different formulations to evaluate tumor growth inhibition using histologic analysis. Mouse brains were fixed with 4% paraformaldehyde. Fixed tissues were cryosectioned and stained with hematoxylin and eosin (H&E). Apoptotic activity was detected via staining using an In Situ Cell Death Detection Kit, POD (Roche) according to the manufacturer's protocol. Nuclei were counterstained with DAPI.

Characterization. Proton NMR spectrum was recorded on a Bruker AVANCE 600 MHz spectrometer. MALDI-TOF mass spectrum was recorded on a Bruker REFLEX-III instrument. Dynamic light scattering (DLS) studies were performed using a Zetatrac (Microtrac Inc.) instrument, and the area-based mean particle sizes were presented. Zeta potential measurements were carried out on a Malvern Nano-ZS zetasizer at room temperature. Transmission electron microscopy (TEM) images were taken on a JEOL JEM-2100 HR instrument operating at a voltage of 200 kV. The samples were prepared by dropping the solutions onto carbon coated grids, and stained by uranyl acetate. UV-vis spectra were recorded on a Thermo Scientific Nanodrop 2000c spectrophotometer.

Statistical Analysis. Data were presented as Mean±SD. Statistical analysis was performed using one-tailed student's t-test. The difference between test groups and control groups were considered statistically significant when P<0.05.

While the disclosure has been described through illustrative examples, routine modifications of the various examples will be apparent to those skilled in the art and such modifications are intended to be within the scope of this disclosure.

1. A lipidic compound-telodendrimer hybrid nanoparticle comprising:
a lipidic compound;
a telodendrimer;
wherein the lipidic compound-telodendrimer hybrid nanoparticle has a size of 1 nm to 100 nm.

2. The lipidic compound-telodendrimer hybrid nanoparticle of claim 1, wherein the lipidic compound is a lipid.

3. The lipidic compound-telodendrimer hybrid nanoparticle of claim 1, wherein the lipidic compound is a lipidoid.

4. The lipidic compound-telodendrimer hybrid nanoparticle of claim 1, wherein the lipidic compound is a lipid and the lipidic compound-telodendrimer hybrid nanoparticle further comprises a small molecule and/or a peptide and/or a protein.

5. The lipidic compound-telodendrimer hybrid nanoparticle of claim 1, wherein the lipidic compound is a lipidoid and the lipidic compound-telodendrimer hybrid nanoparticle further comprises a protein and/or peptide.

6. The lipidic compound-telodendrimer hybrid nanoparticle of claim 1, wherein the telodendrimer is a functional segregated telodendrimer.

7. The lipidic compound-telodendrimer hybrid nanoparticle of claim 1, wherein the telodendrimer comprises one or more poly(ethylene glycol) groups.

8. The lipidic compound-telodendrimer hybrid nanoparticle of claim 1, wherein the lipidic compound-telodendrimer hybrid nanoparticle further comprises cholesterol.

9. A composition comprising one or more lipidic compound-telodendrimer hybrid nanoparticle of claim 1.

10. The composition of claim 9, wherein the composition comprises a lipid-telodendrimer hybrid nanoparticle, a lipidoid-telodendrimer hybrid nanoparticle, or a combination thereof.

11. The composition of claim 9, wherein the one or more lipidic compound-telodendrimer hybrid nanoparticle further comprises a small molecule and/or a peptide and/or a protein.

12. The composition of claim 9, wherein the composition further comprises an aqueous component.

13. The composition of claim 9, wherein the composition further comprises a pharmaceutically acceptable carrier.

14. The composition of claim 9, wherein one or more of the one or more lipidic compound-telodendrimer hybrid nanoparticle further comprises cholesterol.

15. A method of delivering a small molecule and/or protein and/or peptide comprising administering a plurality of lipidic compound-telodendrimer hybrid nanoparticles comprising the small molecule and/or the protein and/or the peptide or a composition comprising a plurality of lipidic compound-telodendrimer hybrid nanoparticles comprising the small molecule and/or the protein and/or the peptide to an individual.

16. The method of claim 15, wherein lipidic compound of the one or more of the lipidic compound-telodendrimer hybrid nanoparticles or one or more of the lipidic compound-telodendrimer hybrid nanoparticles of the composition is a lipid, lipidoid, or a combination thereof.

17. The method of claim 15, wherein the telodendrimer of the one or more of the lipidic compound-telodendrimer hybrid nanoparticles or one or more of the lipidic compound-telodendrimer hybrid nanoparticles of the composition is a functional segregated telodendrimer.

18. The method of claim 15, wherein the one or more of the lipidic compound-telodendrimer hybrid nanoparticles or one or more of the lipidic compound-telodendrimer hybrid nanoparticles of the composition comprise cholesterol.

19. The method of claim 15, wherein the individual is a human.

20. The method of claim 15, wherein the administration is topical, parenteral, intravenous, intradermal, subcutaneous, intramuscular, intratumoral, intercranial, colonical, intraperitoneal, oral, or nasal.


SEQUENCE LISTING (1 sequence)

SEQ ID NO: 1 - protein, 389 aa, Corynebacterium diphtheriae
GADDVVDSSKSFVMENFSSYHGTKPGYVDSIQKGIQKPKSGTQGNYDDDWKGFYSTDNKYDAAGYSVDNENPLSGKAGGVVKVTYPGLTNVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPF